(19) United States
(12) Patent Application Publication
(10) Pub. No.: US 2023/0065757 A1
(43) Pub. Date: Mar. 2, 2023

(54) METHOD FOR PREDICTING PATIENT RESPONSE TO IMMUNOTHERAPY

(71) Applicant: The Board of Trustees of the Leland Stanford Junior University, Stanford, CA (US)

(72) Inventors: Christian M. SCHÜRCH, Stanford, CA (US); Darci J. Phillips, Stanford, CA (US); Garry P. Nolan, Redwood City, CA (US)

(21) Appl. No.: 17/795,216

(22) PCT Filed: Feb. 4, 2021
(86) PCT No.: PCT/US2021/016641

§ 371 Date: Jul. 25, 2022

Related U.S. Application Data
(60) Provisional application No. 62/971,722, filed on Feb. 7, 2020.

(51) Int. Cl. G01N 33/50; C07K 16/28; A61P 35/00; G01N 33/569; G01N 33/574

(52) U.S. Cl. CPC G01N 33/5091; C07K 16/2818; A61P 35/00; G01N 33/56972; G01N 33/57484; C07K 2317/24; A61K 2039/505

(57) ABSTRACT

Provided herein, among other things, is a method for predicting how a patient responds to immunotherapy. In some embodiments, the method may comprise: performing a multiplexed binding assay on a tissue section of a tumor obtained from a cancer patient to identify at least cancer cells, effector immune cells and immunosuppressive cells in the tissue section; measuring, for each cell of a plurality of the effector immune cells: (i) the physical distance to its most proximal cancer cell; and (ii) the physical distances to its most proximal immunosuppressive cell; and calculating, for each of the effector immune cells analyzed, the ratio of the distance measured in step (i) and distance measured in step (ii), wherein the ratio is predictive of the patient's response to immunotherapy. The method may be used to select patients for immunotherapy.

### CROSS-REFERENCING

This application claims the benefit of U.S. provisional application Ser. No. 62/971,722, filed on Feb. 7, 2020, which application is incorporated by reference herein.

### BACKGROUND

Cutaneous T cell lymphoma (CTCL) is a rare, heterogenous group of CD4⁺ T cell malignancies that primarily affect the skin. Advanced stage disease develops in ˜25% of patients¹, whose 5-year survival rate is less than 30%². There are no curative systemic therapies for advanced CTCL, and current treatments usually only induce short-lived, partial disease control³. Immune checkpoint inhibitors, such as antibodies against programmed cell death protein 1 (PD-1), can reinvigorate exhausted, tumor-specific T cells, promoting robust is and durable responses in multiple advanced cancers⁴⁻⁶. In CTCL, PD-1 and its ligands can be expressed on both tumor and reactive immune cells, making this pathway a promising therapeutic target⁷⁻¹⁰.

A multicenter phase II clinical trial of the PD-1-blocking antibody pembrolizumab in advanced relapsed or refractory CTCL was recently reported¹¹. In this study 38% of patients achieved a sustained clinical response, whereas 40% of those who did not respond experienced rapid disease progression. This rapid progression likely results from inhibiting PD-1 on tumor cells and in turn stimulating their growth^{12,13}. These outcomes underscore the need for biomarkers that predict pembrolizumab response in CTCL, which would allow patients to be stratified into probable responders and non-responders prior to initiating therapy. To date, biomarker discovery studies with immunohistochemistry and gene expression profiling of skin biopsies and mass cytometry of peripheral blood have failed to identify predictors of pembrolizumab response in CTCL¹¹.

Better methods for predicting response to immunotherapy are therefore needed.

### SUMMARY

Provided herein, among other things, is a method for predicting how a patient responds to immunotherapy. In some embodiments, the method may comprise performing a multiplexed binding assay on a tissue section of a tumor obtained from a cancer patient to identify at least cancer cells, effector immune cells and immunosuppressive cells in the tissue section; measuring, for each cell of a plurality of the effector immune cells: (i) the physical distance to its most proximal cancer cell; and (ii) the physical distance to its most proximal immunosuppressive cell; and calculating, for each of the effector immune cells analyzed, the ratio of the distance measured in (i) and the distance measured in (ii). This ratio is predictive of the patient's response to immunotherapy and, as such, may be used to select patients for immunotherapy.

These and other aspects of the invention are described in greater detail below.

### BRIEF DESCRIPTION OF THE FIGURES

The skilled artisan will understand that the drawings described below are for illustration purposes only. The drawings are not intended to limit the scope of the present teachings in any way.

FIG. 1. Biomarker discovery in CTCL patients treated with pembrolizumab using is CODEX and RNAseq. a, Experimental design to study 14 advanced CTCL patients treated with pembrolizumab via the CITN-10 clinical trial (ClinicalTrials.gov identifier: NCT02243579), including [1] sample collection, [2] tissue microarray creation, [3] CODEX and RNAseq experiments, and [4] integrative data analysis. b, Kaplan-Meier curves comparing survival in responders (n=7) and non-responders (n=7). c, Immunohistochemistry staining of CD4, FOXP3, PD-1, and PD-L1 in representative CTCL tumors from responders and non-responders before pembrolizumab treatment. d, Bar plot of CD3 (T cells), CD4 (helper T cells), CD8 (cytotoxic T cells), FOXP3 (Tregs), CD163 (M2 macrophages), PD-1, PD-L1, and PD-L2 expression in responders and non-responders pre-treatment, illustrating no differences and failing to identify a predictive biomarker. e, CODEX antibody panel, consisting of 55 tumor, immune, functional and stromal markers. f, Identification of 21 CODEX cell type clusters, including 13 immune cell clusters, 2 tumor cell clusters and 6 auxiliary cell clusters. One of the immune cell clusters was composed of CD4+ T cells. Both tumor cell clusters were composed of CD4+ tumor cells. Using the multiplexed capability of CODEX, we were able to distinguish benign CD4+ T cells from CD4+ tumor cells. g, Visual verification of the assignment of CD4+ T cell (blue cross) and tumor cell (red cross) clusters, with hematoxylin and eosin, DRAQ5 (nuclear stain), CD3, CD4, CD5, CD7, CD25, Ki-67, and PD-1. h, Bar plot of the average expression of Ki-67, CD2, CD3, CD4, CD5, CD7, CD25, CD30, PD-1, and PD-L1 on tumor cells (red bars) relative to CD4+ T cells (blue line), showing that tumor cells have lower expression of CD7 and increased expression of CD25 and Ki-67, consistent with the known phenotype of CTCL cells. i, Plot of medians with 95% confidence intervals showing that the nuclear size of tumor cells (n=38,499, median 4792) is greater than that of CD4+ T cells (n=1647, median 3842), p<0.0001. j, RNAseq identified genes predictive of tumor cells, irrespective of CD4+ T cells.

FIG. 2. Characterization of the CTCL TME pre- and post-pembrolizumab treatment by CODEX. a, A seven color fluorescent CODEX image from a representative responder pre-treatment (left) stained with CD4 (green), CD8 (cyan), FOXP3 (blue), CD68 (magenta), CD31 (white), cytokeratin (yellow) and Ki-67 (red). The corresponding cell type cluster map (right) highlights helper T cells and tumor cells (green), cytotoxic T cells (cyan), Tregs (blue), macrophages (magenta), blood vessels (white), epithelium (yellow), and proliferating T cells/tumor cells (red). Excellent correlation between the CODEX image and cell type cluster map is appreciated. The corresponding H&E image is shown as insert within the CODEX panel. b, A seven color fluorescent CODEX image from a representative non-responder pre-treatment (left) and corresponding cell type cluster map (right), using the same color scheme as panel a. The corresponding H&E image is shown as insert within the CODEX panel. c, The combined frequencies of tumor, immune and auxiliary cell types are evenly distributed between groups (left). The combined frequencies of CD4+ T cells, CD8+ T cells, Tregs, M1 macrophages, M2 macrophages, and other pooled immune cells (right). d, Bar plots of CD4+ T cells, CD8+ T cells, Tregs, M1 macrophages, and M2 macrophages as a percentage of all immune cells in responders and non-responders before and after treatment. Like the baseline immunohistochemistry bar plots above (FIG. 1d), no differences are appreciated based solely on the frequency of cell types.

FIG. 3. Cellular neighborhoods reveal differences in the spatial TME organization in responders and non-responders. a, Cellular neighborhood (CN) analysis schematic. [1] Selection of computational parameters, including the window size (five in this schematic) and the number of CNs to be computed (five in this schematic). [2] Assignment of an index cell (i) to a given CN based on the composition of cell types of its five nearest neighbors. [3] After clustering of the index cells, CNs are determined and represented as a heatmap. [4] Visualization of CNs as a Voronoi diagram. b, Identification of 10 CNs in the CTCL TME, which were conserved across patient groups. c, When visualized as Voronoi diagrams, these 10 CNs recapitulate well-defined architectural features in a responder post-treatment (left) and non-responder post-treatment (right). d, Corresponding H&E and seven color fluorescent CODEX images (same markers shown in FIG. 2a-b), confirm the CN assignments. e, CN frequency differences in CN-2, CN6 and CN-9 across patient groups before and after pembrolizumab treatment.

FIG. 4. Spatial relationship between PD-1+CD4+ T cells, Tregs and tumor cells predicts pembrolizumab response in CTCL. a, SpatialScore schematic (left), whereby the Euclidean distances between every PD-1+CD4+ T cell and its nearest tumor cell (distance A) and every PD-1+CD4+ T cells and its nearest Treg (distance B) is measured. The SpatialScore is then computed by taking the ratio of distance A/distance B. SpatialScore interpretation (right), whereby a lower SpatialScore indicates increased anti-tumor activity (i.e., PD-1+CD4+ T cells are closer to tumor cells and farther from Tregs) and a higher SpatialScore indicates increased immunosuppression (i.e., PD-1+CD4+ T cells are closer to Tregs and father from tumor cells). b, Plot of means of the SpatialScore of pooled cells for responders and non-responders before and after pembrolizumab treatment.

FIG. 5. CXCL13 is a key driver of pembrolizumab response in CTCL. a, Seven genes predictive of the SpatialScore identified by RNAseq. b, CXCL13 expression across groups. c, CXCL13 expression on a per patient before and after treatment in responders (left) and non-responders (right). CXCL13 expression increased in 5/5 (100%) responder patients, in contrast to 1/6 (16.7%) in non-responder patients. d, CXCR5, which is the receptor for CXCL13, expression across groups. While not statistically significant, the trend for CXCR5 expression mirrors that of CXCL13.

FIG. 6. Model for pembrolizumab responders and non-responders in CTCL.

### DEFINITIONS

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of ordinary skill in the art with the general meaning of many of the terms used herein. Still, certain terms are defined below for the sake of clarity and ease of reference.

A “plurality” contains at least 2 members. In certain cases, a plurality may have at is least 2, at least 5, at least 10, at least 100, at least 1000, at least 10,000, at least 100,000, at least 10⁶, at least 10⁷, at least 10⁸ or at least 10⁹ or more members. In certain cases, a plurality may have 2 to 100 or 5 to 100 members.

As used herein, the term “labeling” refers to a step that results in binding of a binding agent to specific sites in a sample (e.g., sites containing an epitope for the binding agent (e.g., an antibody) being used, for example) such that the presence and/or abundance of the sites can be determined by evaluating the presence and/or abundance of the binding agent. The term “labeling” refers to a method for producing a labeled sample in which any necessary steps are performed in any convenient order, as long as the required labeled sample is produced. For example, in some embodiments and as will be exemplified below, a sample can be labeled using a plurality of binding agents that are each linked to an oligonucleotide.

As used herein, the term “tissue section” refers to a piece of tissue that has been obtained from a subject, fixed, sectioned, and mounted on a planar surface, e.g., a microscope slide or coverslip. In particular embodiments, the tissue section may be a section of a tissue biopsy obtained from a patient. Biopsies of interest include both tumor and non-neoplastic biopsies of skin (melanomas, carcinomas, lymphomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc.

As used herein, the term “formalin-fixed paraffin embedded (FFPE) tissue section” refers to a piece of tissue, e.g., a biopsy sample that has been obtained from a subject, fixed in formalin, embedded in wax, cut into thin sections, and then mounted on a microscope slide.

Other definitions of terms may appear throughout the specification.

### DETAILED DESCRIPTION

As noted above, provided herein, among other things, is a method for predicting how a patient responds to immunotherapy. In some embodiments, the method may comprise performing a multiplexed binding assay on a tissue section of a tumor obtained from a cancer patient to identify at least cancer cells, effector immune cells and immunosuppressive cells in the tissue section; measuring, for each cell of a plurality of the effector immune cells: (i) the physical distance to its most proximal cancer cell; and (ii) the physical distance to its most proximal immunosuppressive cell; and calculating, for each of the effector immune is cells analyzed, the ratio of the distance measured in (i) and distance measured in (ii).

As demonstrated below, the ratio calculated in the method is predictive of the patient's response to immunotherapy. For example, a smaller ratio (e.g., a ratio that is 0.5 or below) indicates that the patient will have a better response to immunotherapy.

The cancer cells identified in the method may be of any type of cancer for which a treatment by immunotherapy exists. For examples, the cancer cells identified in the method may be melanoma cells, carcinoma cells, lymphoma cells, sarcoma cells or glioma cells. For example, the cancer may be melanoma, lung cancer, breast cancer, head and neck cancer, bladder cancer, Merkel cell cancer, cervical cancer, hepatocellular cancer, gastric cancer, cutaneous squamous cell cancer, classic Hodgkin lymphoma, B-cell lymphoma, colorectal carcinoma, pancreatic carcinoma, gastric or breast carcinoma, for which the markers are known.

Immunological markers to identify cancer cells are generally well known and available for many types of cancers (see, generally, Painter et al, Toxicol. Pathol. 2010 38: 131-141 and Bahrami et al Arch Pathol Lab Med. 2008 132:326-48, among many others). For example, the cancer cells identified in step (a) may be i. melanoma cells identified by expression of one or more of the following markers: S-100, Melan-A, Sox10, MITF, tyrosinase, and HMB45 (e.g., S-100, Melan-A, Sox10 and HMB45); ii. carcinoma cells identified by the expression of one or more of the following markers: pan-cytokeratin (CK), CK7, CK20, CK5/6, CK8/18, napsin A, TTF-1, PSA, PSMA, CDX2, GATA3, synaptophysin, chromogranin A, NSE, EpCAM, and MUC-1 (e.g., CK7, CK2n, TTF-1, PSA, CDX2, GATA3); iii. lymphoma/leukemia cells identified by the expression of one or more of the following markers: CD45, CD3, PAX5, CD20, Myc, CyclinDm, BCL-2, BCL-6, RF4, CD138, CD30, kappa, lambda, TdT, CD16, ALK, and lysoszyme (e.g., CD45, PAX5, rCD2k, Myc, CyclinD1, BCL-2, BCL-6, CRF4, CD138, and CD3); iv. sarcoma/mesothelioma cells identified by the expression of one or more of the following markers: vimentin, SMA, desmin, caldesmin, MyoD, CD34, calretinin, podoplanin, and CD47 (e.g., vimentin, SMA, desmin, CD34); v. glioma cells/neural tumor cells identified by the expression of one or more of the following markers: GFAP, DH-1(R132H), 10 neurofilament, and NeuN (e.g, GFAP, DH-1(R132H)); or vi. germ cell tumor cells identified by the expression of one or more of the following markers: beta-HCG, OCT4, SALL4, PLAP, inhibin A, HPL and AFP.

Exemplary panels of markers for identifying various cancer cells are shown in the following table, although there are many alternatives that can be used.

| | |
|---|---|
| Acute Leukemia IHC Panel | CD3, CD7, CD10, CD20, CD34, CD45, CD56, CD61, |
| | CD71, CD117, MPO, PAX-5, and TdT. |
| Adenocarcinoma vs. Mesothelioma IHC | Pan-CK, CEA, MOC-31, BerEP4, TTF1, calretinin, and WT- |
| Panel | 1. |
| Bladder vs. Prostate Carcinoma IHC | CK7, CK20, PSA, CK 903, uroplakin, thrombomodulin, p53 |
| Panel | and p63. |
| Breast IHC Panel | ER, PR, Ki-67, and HER2. Reflex to HER2 FISH after |
| | HER2 IHC is available. |
| Burkitt vs. DLBC Lymphoma IHC panel | BCL-2, c-MYC, Ki-67. |
| Carcinoma Unknown Primary Site, | CK7, CK20, mammaglobin, GATA-3, ER, TTF1, CEA, |
| Female (CUPS IHC Panel - Female ) | CA19-9, S100, synaptophysin, and WT-1. |
| Carcinoma Unknown Primary Site, Male | CK7, CK20, TTF1, PSA, CEA, CA19-9, S100, and |
| (CUPS IHC Panel - Male) | synaptophysin. |
| GIST IHC Panel | CD117, DOG-1, CD34, and desmin. |
| Hepatoma/Cholangio vs. Metastatic | HSA (HepPar 1), CDX2, CK7, CK20, CAM 5.2, TTF-1, and |
| Carcinoma IHC Panel | CEA (polyclonal). |
| Hodgkin vs. NHL IHC Panel | BOB-1, BCL-6, CD3, CD10, CD15, CD20, CD30, CD45 |
| | LCA, CD79a, MUM1, OCT-2, PAX-5, and EBER ISH. |
| Lung Cancer IHC Panel | chromogranin A, synaptophysin, CK7, p63, and TTF-1. |
| Lung vs. Metastatic Breast Carcinoma | TTF1, mammaglobin, GCDFP-15 (BRST-2), and ER. |
| IHC Panel | |
| Lymphoma Phenotype IHC Panel | BCL-2, BCL-6, CD3, CD4, CD5, CD7, CD8, CD10, CD15, |
| | CD20, CD30, CD79a, CD138, cyclin D1, Ki67, MUM1, |
| | PAX-5, TdT, and EBER ISH. |
| Lymphoma vs. Carcinoma IHC Panel | CD30, CD45, CD68, CD117, pan-keratin, MPO, S100, and |
| | synaptophysin. |
| Lymphoma vs. Reactive Hyperplasia IHC | BCL-2, BCL-6, CD3, CD5, CD10, CD20, CD23, CD43, |
| Panel | cyclin D1, and Ki-67. |
| Melanoma vs. Squamous Cell Carcinoma | CD68, Factor XIIIa, CEA (polyclonal), S-100, melanoma |
| IHC Panel | cocktail (HMB-45, MART-1/Melan-A, tyrosinase) and Pan- |
| | CK. |
| Mismatch Repair Proteins IHC Panel | MLH1, MSH2, MSH6, and PMS2. |
| (MMR/Colon Cancer) | |
| Neuroendocrine Neoplasm IHC Panel | CD56, synaptophysin, chromogranin A, TTF-1, Pan-CK, |
| | and CEA (polyclonal). |
| Plasma Cell Neoplasm IHC Panel | CD19, CD20, CD38, CD43, CD56, CD79a, CD138, cyclin |
| | D1, EMA, IgG kappa, IgG lambda, and MUM1. |
| Prostate vs. Colon Carcinoma IHC Panel | CDX2, CK 20, CEA (monoclonal), CA19-9, PLAP, CK 7, |
| | and PSA. |
| Soft Tissue Tumor IHC Panel | Pan-CK, SMA, desmin, caldesmon, MyoD1, myogenin, |
| | S100, CD34, vimentin, and CD68. |
| T-Cell Lymphoma IHC panel | ALK1, CD2, CD3, CD4, CD5, CD7, CD8, CD10, CD20, |
| | CD21, CD30, CD56, TdT, and EBER ISH. |
| T-LGL Leukemia IHC panel | CD3, CD8, granzyme B, and TIA-1. |
| Undifferentiated Tumor IHC Panel | Pan-CK, CK8/18, S100, CD45, and vimentin. |

The effector immune cells identified in the method include one or more of CD4+ T cells, CD8+ T cells, gamma-delta T cells, NK cells, NK T cells and M1 macrophages. In many embodiments, all of these effector immune cell types are detected. For example, in some embodiments, the effector immune cells identified in the method may include: i. CD4+ T cells identified by the expression of CD3, CD4, and TCR-a/b; ii. CD8+ T cells identified by the expression of CD3, CD8, TCR-a/b; iii. gamma-delta T cells identified by the expression of CD3, and TCR-g/d; iv. NK cells identified by the expression of CD16 and CD56; v. NK T cells identified by the expression of CD3, CD16, and CD56; and vi. M1 10 macrophages identified by the expression of CD68. Other markers for such cells and other types of effector immune cell types may become known.

The immunosuppressive cells identified the method include regulatory T cells, M2 macrophages, and N2 granulocytes. In many embodiments, all of these immunosuppressive cells types are detected. For example, in some embodiments, the immunosuppressive cells 15 identified in the method include: i. regulatory T cells identified by the expression of FoxP3; ii. M2 macrophages identified by the expression of CD163 and CD206; and iii. N2 granulocytes identified by the expression of CD15 and MMP9.

For example, at a minimum, the plurality of binding agents used in the multiplex binding assay may comprise binding agents that specifically bind to CD3, CD4, CD8, TCR-g/d, CD16, CD56, CD68 (for effector immune cells) and FoxP3, CD163, CD206, CD15, MMP9 (for immunosuppressive cells), as well as binding agents that recognize the cancer cells.

Other types of cells (in addition to the cancer cells, effector immune cells and immunosuppressive cells) may be detected in the assay. For example, fibroblasts, pericytes, dendritic cells, endothelial cells, bone cells, muscle cells, fat cells, skin cells, nerve cells, and neuroendocrine cells may also be identified, depending on the tissue. Methods for labeling such cells are well known.

Moreover, in addition, other functional markers may be analyzed (e.g., PD-1, PD-L1, CTLA-4, ICOS, LAG-3, TIM-3, VISTA etc.). These markers may indicate which type of immunotherapy or combination immunotherapy should be administered to the patient.

The multiplexed binding assay may be done by detecting binding of at least 10, at least 20, at least 50, up to 100 or 300 binding agents (e.g., antibodies) to a tissue section, e.g., a crosslinked tissue section such as an FFPE section. Methods for performing multiplexed binding assay include, but are not limited to, multiplex colorimetric immunohistochemistry (mCIHC), multiplex immunofluorescence (mIF), cyclic immunofluorescence (CycIF), iterative indirect immunofluorescent imaging (4i), imaging mass cytometry (IMC), multiplexed ion beam imaging (MIBI), codetection by indexing (CODEX), and digital spatial profiling (DSP). These methods are reviewed in, e.g., Patel et al (Methods Mol Biol. 2020 2055:455-465) and Francisco-Cruz et al (Methods Mol. Biol. 2020 2055: 467-495). The general principles of CycIF are described in Rashid et al. Sci. Data. 2019 6: 323 and Lin et al. eLife 2018 10: 31657. The general principles of 4i are described in Gut et al (Science 2018 361: 1-13). The general principles of IMC and MIBI, which are mass-spectrometry approaches for performing multiplexed tissue labeling, are described in a variety of publications including, but not limited to Angelo et al. (Nature Medicine 2014 20:436), Rost et al. (Lab. Invest. 2017 97: 992-1003) U.S. Pat. Nos. 9,766,224, 9,312,111 and US2015/0080233, among many others.

The general principles of CODEX are described in Goltsev et al. (Cell 2018 174: 968-981), SchUrch et al. (bioRxiv 2019 743989) and US20180030504. CODEX-based implementations of the method may involve (a) obtaining: i. a plurality of capture agents (e.g., 20-200 antibodies) that are each linked to a different oligonucleotide; and ii. a corresponding plurality of labeled nucleic acid probes, wherein each of the labeled nucleic acid probes specifically hybridizes with only one of the oligonucleotides; (b) labeling the sample with the plurality of capture agents; (c) specifically hybridizing a first sub-set (e.g., 2, 3, or 4) of the labeled nucleic acid probes of (a)(ii) with the sample, wherein the probes in the first sub-set are distinguishably labeled, to produce labeled probe/oligonucleotide duplexes; (d) reading the sample to obtain an image showing binding pattern for each of the probes hybridized in step (c); (e) inactivating or removing the labels that are associated with the sample in step (c), leaving the plurality of capture agents of (b) and their associated oligonucleotides still bound to the sample; and (f) repeating steps (c) and (d) multiple times with a different sub-set of the labeled nucleic acid probes of (a)(ii), each repeat followed by step (e) except for the final repeat, to produce a plurality of images of the sample, each image corresponding to a sub-set of labeled nucleic acid probes used in (c). In these embodiments, multiple images may be registered and superimposed. Using any method, the is image(s) provide information on the amount of each antibody that is bound to the sample as well as the location of the epitope to which it binds.

After imaging, the cells in the image can be segmented, meaning that the boundaries or edges of the cells are defined. In some cases, image segmentation may be done for only the cancer cells, effector immune cells and immunosuppressive cells. However, in other embodiments, all cells in the image may be segmented. Image segmentation may by any a variety of techniques. In some embodiments, the cells may be segmented using a watershed algorithm (see, e.g., Al-Lofahi et al. BMC Bioinformatics. 2018 19: 365) although many other may be used. In watershed-based segmentation, the contents of each cell's nucleus are identified by a nuclear staining, such as by DRAQ-5 or Hoechst. The watershed algorithm identifies each nucleus and draws a border around it, allowing cells to be detected and touching cells to be separated. In addition to the watershed algorithm, other algorithms include manual tracing of cells, levelset method, morphology-based segmentation, active contours model, snake algorithm, and more recently, deep learning techniques. Segmentation methods that can be used to define the edges of cells in highly multiplexed tissue images are described in a variety of publications, including Schuffler et al. (Cytometry A. 2015 87: 936-42) and Wang et al. (Am J Pathol. 2019 189:1686-1698). After segmentation, the binding pattern of the binding agents to the cells (which, in turn, reflects the presence and abundance of the epitopes to which agents bind) are used to discriminate the cancer cells, effector immune cells and immunosuppressive cells, and, if desirable other (e.g., stromal) cell types and compute their numbers and distributions within tumors and surrounding normal tissue.

After the cells are segmented and the cell types are identified, the distances between the cells can be measured. As noted above, this step may be done by measuring, for each cell of a plurality of the effector immune cells (e.g., at least 100, at least 500, at least 1,000, at least 2,000 or more effector immune cells): (i) the physical distance to its most proximal cancer cell (i.e., the cancer cell that is closest to each of the effector immune cells); and (ii) the physical distance to its most proximal immunosuppressive cell (i.e., the immunosuppressive cell that is closest to each of the effector immune cells). Other distances may also be measured. In practice, many cell types (e.g., tumor cells, effector T cells (CD4+ T cells, CD8+ T cells), M1 macrophages, immunosuppressive cells (Tregs, M2 macrophages), dendritic cells, stromal cells, endothelial cells, etc.) are identified by unsupervised machine learning algorithms (i.e., clustering) followed by supervised confirmation based on marker profile and morphology, and, for each cell of a particular type is (i.e., CD4+ T cells) the closest distance to a cell of a different type (i.e., tumor cell, CD8+ T cell, M1 macrophage, Treg, M2, macrophage, dendritic cell, stromal cell, endothelial cell, etc.) may be calculated. In these embodiments, only the distances between the cells of interest (i.e., tumor cells, specific effector immune cells, specific immunosuppressive cells) are carried forward into the next step of the method.

In some embodiments of the method, the distances between two cells may be calculated by defining the position the cells by a single x-y coordinate, and then measuring the straight line distance between the x-y coordinates of different cells (i.e., by calculating the Euclidean distance between the two points). The position of a cell can be defined in many different ways. For example, the position of a cell can be defined by its centroid (as measured by the plumb line method or balancing method), or by the centroid of its nucleus, although other methods are possible.

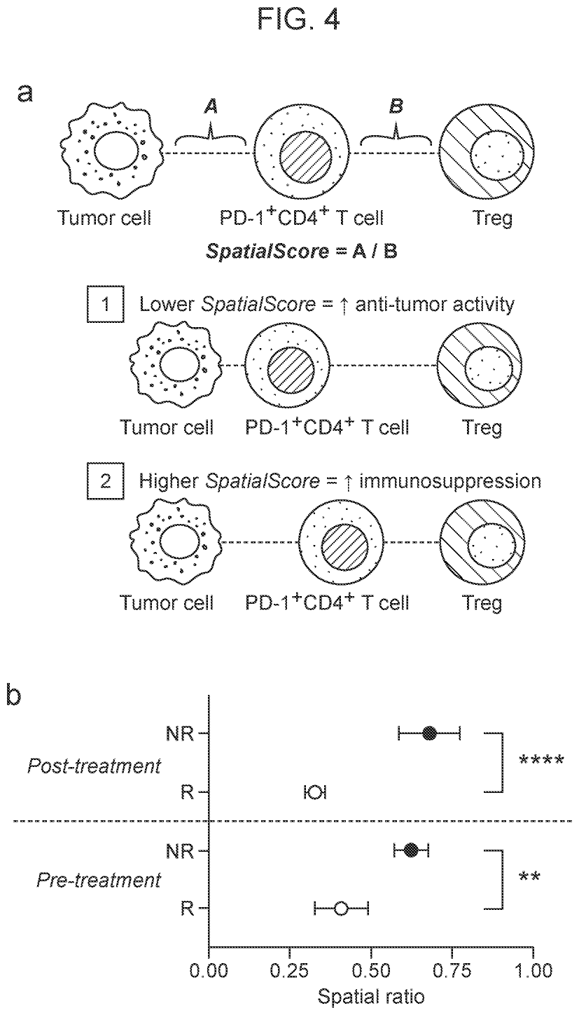

In the next step of the method a ratio may be calculated for each of the effector immune cells analyzed in the method. For each effector immune cell, the ratio is of (i) the physical distance to its most proximal cancer cell (i.e., the cancer cell that is closest to each of the effector immune cells) and (ii) the physical distance to its most proximal immunosuppressive cell (i.e., the immunosuppressive cell that is closest to each of the effector immune cells). For example, if the physical distance of an effector immune cell to its most proximal cancer cell is 10 μm and (ii) the physical distance of that effector immune cell to its most proximal immunosuppressive cell is 10 μm, then the ratio may be 1. However, if the physical distance of an effector immune cell to its most proximal cancer cell is 5 μm and (ii) the physical distance of that effector immune cell to its most proximal immunosuppressive cell is 20 μm, then the ratio may be 0.25. This concept is illustrated in FIG. 4,a.

In some embodiments, this analysis may result in at least 100, at least 500, at least 1,000, at least 2,000 or potentially at least 5,000 ratios where each ratio is for a different effector immune cell and each ratio indicates relative distance between an effector immune cell and the cancer cell that is closest to it and the immunosuppressive cell that is closest to it. As noted above and below, this ratio correlates with response to immunotherapy and can be used to select patients for such treatment. In some embodiments, the ratios may be combined (e.g., averaged, potentially after outliers have been removed) to obtain a score, where the score can be compared to a threshold in order to determine if a patient should receive immunotherapy. For example, in some embodiments, the ratios may be averaged to produce a score, and the score may be compared to a threshold, and an immune checkpoint is inhibitor may be administered to the patient if the score is at or below a threshold. Generally, tumors in which the effector immune cells are closer to cancer cells than they are to immunosuppressive cells are more responsive to immunotherapy.

The immunotherapy may be an immune checkpoint inhibitor such as an antibody that binds to CTLA-4, PD1, PD-L1, TIM-3, VISTA, LAG-3, IDO or KIR. In these embodiments, the immune checkpoint inhibitor may be an antibody, e.g., an anti-CTLA-4 antibody, anti-PD1 antibody, an anti-PD-L1 antibody, an anti-TIM-3 antibody, an anti-VISTA antibody, an anti-LAG-3 antibody, an anti-IDO antibody, or an anti-KIR antibody, although others are known, where the term “antibody” is intended to include nanobodies, phage display antibodies, single chain antibodies, bi-specifics, etc.). In some embodiments, the immunotherapy may also include a co-stimulatory antibody such as an antibody against CD40, GITR, OX40, CD137, or ICOS, for example. In some embodiments, the antibody may be an anti-PD-1 antibody, an anti-PD-L1 antibody or an anti-CTLA-4 antibody. Examples of such antibodies include, but are not limited to: Ipilimumab (CTLA-4), Nivolumab (PD-1), Pembrolizumab (PD-1), Atezolizumab (PD-L1), Avelumab (PD-L1), and Durvalumab (PD-L1). These therapies may be combined with one another and with other therapies. In some embodiments, the dose administered may be in the range of 1 mg/kg to 10 mg/kg, or in the range of 50 mg to 1.5 g every few weeks (e.g., every 3 weeks), depending on the weight of the patient. In certain embodiments, the patient will be treated with the immune checkpoint inhibitor without knowing the PD1, CTLA-4, TIM-3, VISTA, LAG-3, IDO or KIR status of the tumor. However, as noted above, in some cases the tissue section may be stained for PD1, CTLA-4, TIM-3, VISTA, LAG-3, IDO and/or KIR and, as such, the immune checkpoint inhibitor may be selected based on those results. For example, the patient may be identified as having a tumor that contains cells which are positive for one or more of the markers, CTLA-4, PD1, PD-L1, TIM-3, VISTA, LAG-3, IDO or KIR. In these embodiments, if a tumor or the tumor-infiltrating immune cells, are PD-L1 positive, and the ratio is at or below a threshold, then the method may involve administering an anti-PD1 or anti-PD-L1 antibody to the patient. The same principle can be applied to tumors in which cells are positive for other markers.

If the ratio is above the threshold, then the patient may not respond to immunotherapy and, as such an alternative therapy may be administered to the patient. In some cases, the alternative therapy may be a non-targeted therapy, i.e., a therapy that is not targeted to a particular sequence variation. Non-targeted therapies include radiation therapy, systemic or local chemotherapy, hormone therapy, and surgery. Examples of systemic chemotherapies include platinum-based doublet chemotherapy such as the combination of cisplatin and pemetrexed and the combination of cisplatin and gemcitabine. In other cases, the alternative therapy may be a therapy that is targeted to an actionable sequence variation, i.e., a therapy that targets the activity of the protein having a causative sequence variation, where the term “actionable sequence variation” is a sequence variation for which there is a therapy that specifically targets the activity of the protein having the variation. In many embodiments an actionable sequence variation causes an increase in an activity of the protein, thereby resulting in cells containing the variation to grow, divide and/or metastasize without check and in combination with other variations, such as in tumor suppressor genes, leading to cancer. Therapy that is targeted to an actionable sequence variation often inhibits an activity of the mutated protein. Examples of actionable sequence variations are known. For example, targeted therapies directed against these activating alterations in EGFR, ALK, ROS1 and BRAF have been approved for use in patients harboring these activating mutations and fusions, and thus, these are described as “actionable” mutations, although others are known.

In some embodiments, the tissue section may be analyzed at a remote location, potentially by a third party, and the treatment decision may be made upon receipt of a report produced at the remote location and forwarded to a medical professional.

In these embodiments, the method may comprise (a) receiving a report that provides a score indicating the ratio of the physical distances of effector immune cells to their most proximal cancer cell in a tumor from a patient relative to the physical distances of the effector immune cells to their most proximal immunosuppressive cell in the tumor; and (b) identifying the patient as a candidate for immunotherapy if the ratio is at or below a threshold.

In some embodiments, the method may be for selecting a patient for treatment by an immune checkpoint inhibitor. In these embodiments, the method may comprise selecting a cancer patient for treatment by an immune checkpoint inhibitor based on the ratio of the physical distances of effector immune cells to their most proximal cancer cell in a tumor from the patient relative to the physical distances of the effector immune cells to their most proximal immunosuppressive cell in the tumor.

In these embodiments, the report may be in an electronic form, and the method comprises forwarding the report to a remote location, e.g., to a doctor or other medical professional to help identify a suitable course of action, e.g., to identify a suitable therapy for the subject. The report may be used along with other metrics to determine whether the is subject is responsive to a therapy, for example. In some cases, the report may indicate a score as discussed above (such as the “SpatialScore” as described below) as well as a threshold at or below which a patient should be recommend for immunotherapy. For example, the report may indicate a score (e.g., 0.2, 1.0 or 1.5) as well as the threshold (e.g., 0.5) at or below which the patient is likely respond to immunotherapy. The doctor or other medical professional can review the report and make a treatment decision after reviewing the report.

In any embodiment, a report can be forwarded to a “remote location”, where “remote location,” means a location other than the location at which the sequences are analyzed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet, including email transmissions and information recorded on websites and the like. In certain embodiments, the report may be analyzed by an MD or other qualified medical professional, and a report based on the results of the analysis of the sequences may be forwarded to the patient from which the sample was obtained.

In some embodiments, a sample may be collected from a patient at a first location, e.g., in a clinical setting such as in a hospital or at a doctor's office, and the sample may be forwarded to a second location, e.g., a laboratory where it is processed and the above-described method is performed to generate a report. A “report” as described herein, is an electronic or tangible document which includes report elements that provide test results, including the ratio and optionally the threshold. Once generated, the report may be forwarded to another location (which may be the same location as the first location), where it may be interpreted by a health professional (e.g., a clinician, a laboratory technician, or a is physician such as an oncologist, surgeon, pathologist or virologist), as part of a clinical decision.

The results provided by this method may be diagnostic, prognostic, theranostic and, in some cases, may be used to monitor a treatment. In the latter embodiments, ratio may be analyzed at multiple time points in the same patient. In some embodiments, a decrease in the ratio may indicate that a treatment is working and should therefore be continued. In some embodiments, an increase in the ratio, may indicate that a treatment is not working and should therefore be modified or stopped.

As would be readily appreciated, many steps of the method, e.g., image analysis, segmentation, cell identification, centroid identification and distance measurements can be implemented on a computer. As would be apparent, the computational steps described may be computer-implemented and, as such, instructions for performing the steps may be set forth as programing that may be recorded in a suitable physical computer readable storage medium.

The method described above finds particular utility in examining samples using a plurality of antibodies, each antibody recognizing a different marker. Examples of cancers, and biomarkers that can be used to identify those cancers, are shown below. In these embodiments, one does not need to examine all of the markers listed below in order to make a diagnosis.

### Examples

In order to further illustrate some embodiments of the present invention, the following specific examples are given with the understanding that they are being offered to illustrate examples of the present invention and should not be construed in any way as limiting its scope.

CTCL is a malignant CD4+ T cell malignancy of the skin for which the treatment options are limited. CODEX (CO-Detection by indEXing) multiplexed imaging was combined with and RNAseq to deeply phenotype highly infiltrated skin tumors in 14 high-risk, therapy refractory CTCL patients treated with pembrolizumab in a clinical trial. A cellular niche enriched for CD4+ T cells and tumor cells in responders post-treatment and one enriched for Tregs in non-responders before and after treatment were identified. These is differences in spatial organization and functional immune status led us to focus on a simplified spatial relationship between tumor cells, PD-1+CD4+ T cells and Tregs. In doing so, the SpatialScore was developed, defined as the ratio of Euclidean distances between a given PD-1+CD4+ T cell and its nearest tumor cell relative to its nearest Treg cell. When <0.5, the SpatialScore is predictive of a good clinical outcome to anti-PD-1 immunotherapy.

### Methods

Human subjects and clinical trial study design. Cancer Immunotherapy Trials Network-10 (CITN-10) is a multicenter, phase II, single-arm trial that investigated the efficacy of pembrolizumab in 24 patients with two common forms of relapsed or refractory CTCL, mycosis fungoides (MF) and Sezary syndrome (SS)¹¹. CITN-10 obtained written informed consent from all clinical trial participants. The use of tissues for this study was approved by the Stanford University Administrative Panels on Human Subjects in Medical Research (HSR 46894).

All patients had a clinicopathological diagnosis of MF or SS (clinical stage IB to IV) that had relapsed, was refractory to, or had progressed after at least one standard systemic therapy. Exclusion criteria included central nervous system disease, active autoimmune disease, previous exposure to any anti-PD-1, anti-PD-L1, or anti-PD-L2 therapy, and treatment with radiotherapy or other anti-cancer agents within 2 to 15 weeks of the first skin biopsy. Patients were treated with pembrolizumab IV 2 mg/kg every 3 weeks for up to 24 months¹¹. Responses and primary end point (overall response rate) were assessed by consensus global response criteria³⁷.

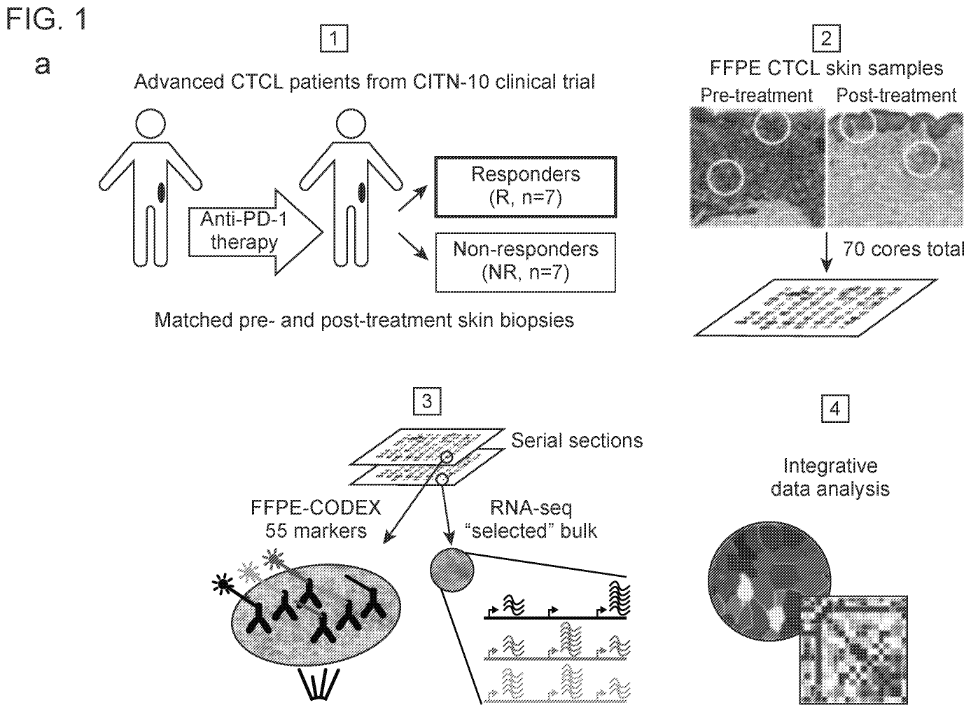

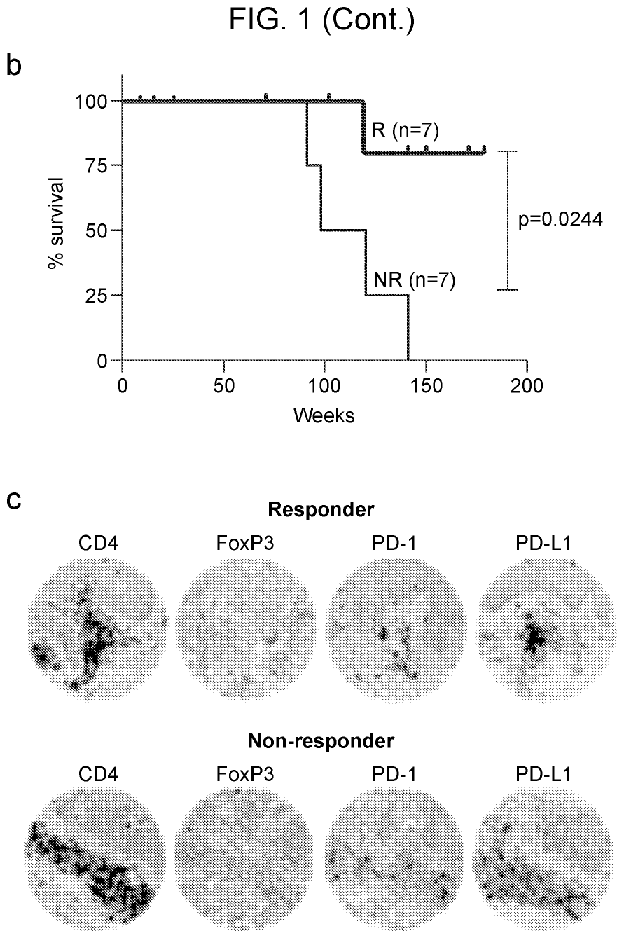

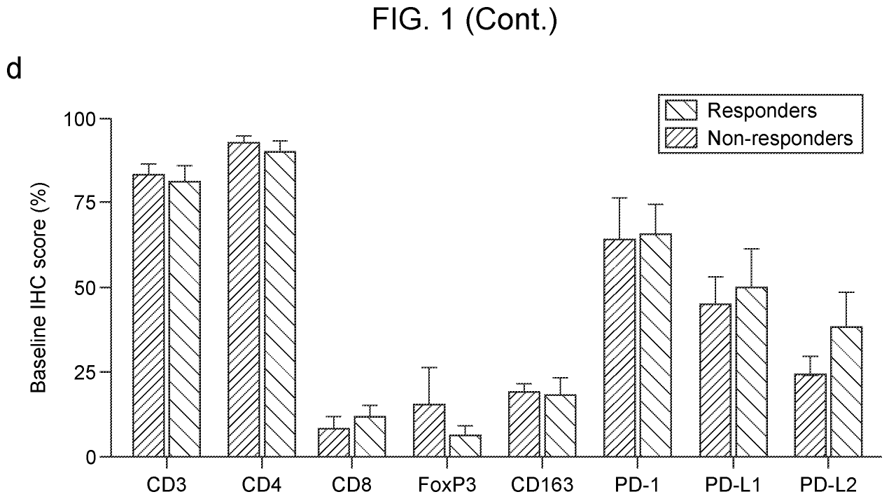

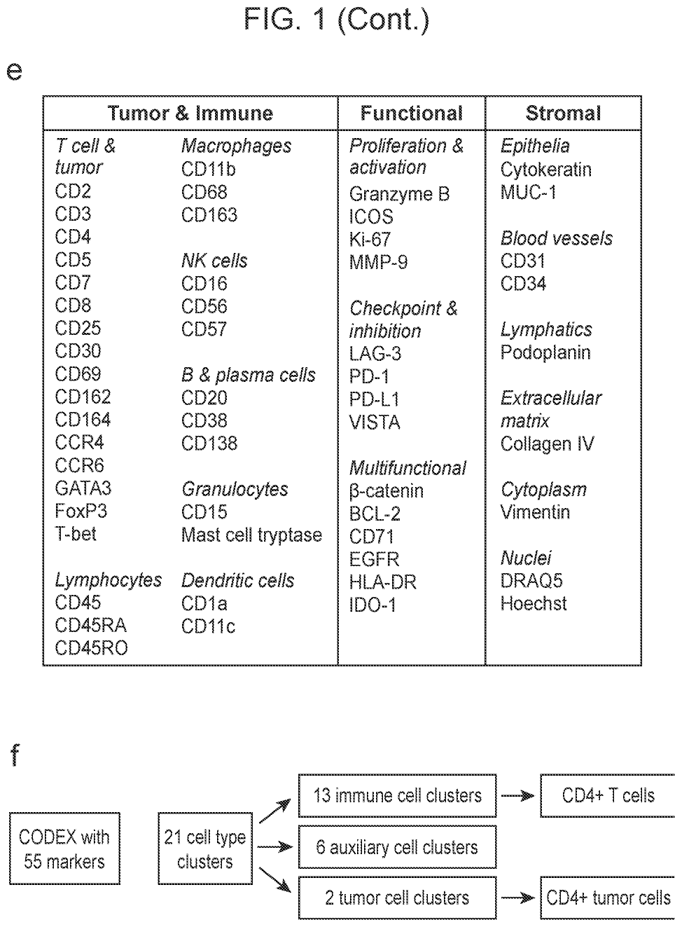

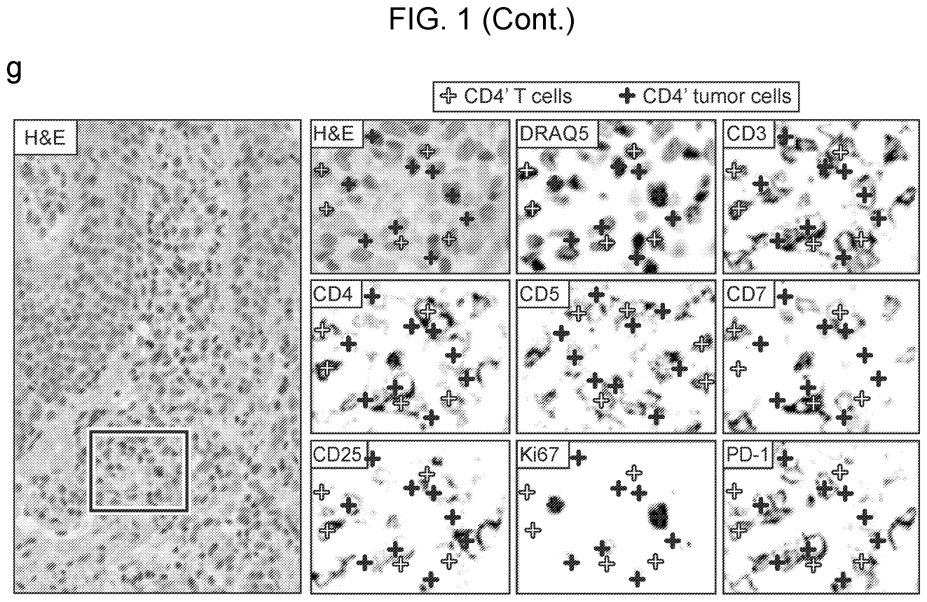

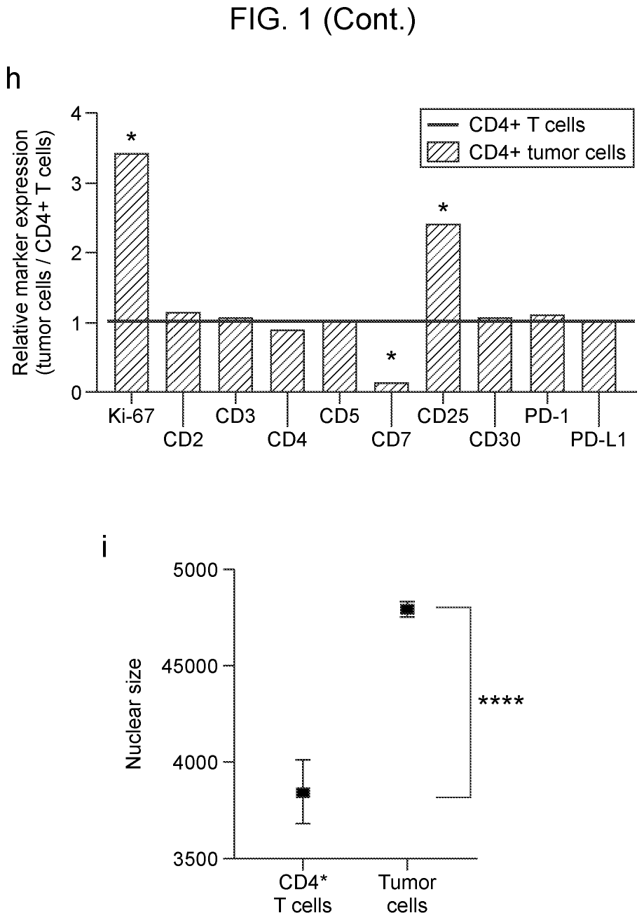

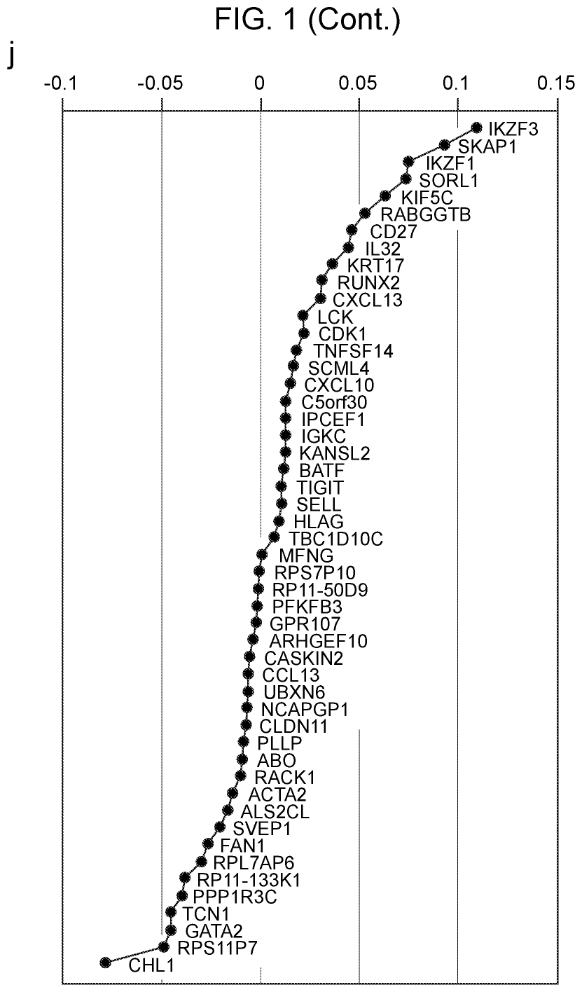

Sample collection and tissue microarray construction. Skin biopsy specimens were collected from the primary tumor site, fixed in formalin and embedded in paraffin. Baseline biopsies were collected prior to pembrolizumab treatment and then at various timepoints during treatment (FIG. 1). Hematoxylin and eosin (H&E) stained biopsy sections from all patients and timepoints were reviewed by two expert pathologists (C.M.S. and R.P.). Fourteen of the 24 biopsy samples had adequate FFPE material, and two to three 0.6 mm cores from the most infiltrated regions for each patient and timepoint were digitally annotated and compiled into a formalin-fixed, paraffin-embedded (FFPE) tissue microarray. The tissue microarray was sectioned at 4 μm thickness and mounted onto Vectabond™ (Vector Labs)-pre-treated square glass coverslips (22×22 mm, Electron Microscopy Sciences).

Immunohistochemistry. Immunohistochemistry (IHC) for CD3 (clone CD3-12; Abd is Serotec), CD4 (clone 4B12; Leica), CD8 (clone CD8/144B; Dako), FoxP3 (clone 236A/E7; Abcam), CD163 (clone 10D6; Thermo Fisher), PD-1 (clone NAT105; Cell Marque), PD-L1 (clone 22C3; Merck Research Laboratories), and PD-L2 (clone 3G2; Merck Research Laboratories) was performed as previously described³⁸. IHC was graded according to the positive percentage of the total mononuclear cell infiltrate¹¹.

Antibodies. For CODEX, purified, carrier-free monoclonal and polyclonal anti-human antibodies were purchased from commercial vendors. Conjugations to maleimide-modified short DNA oligonucleotides (TriLink) were performed at a 2:1 weight/weight ratio of oligonucleotide to antibody, with at least 100 μg of antibody per reaction, as previously described by Schurch et al. (bioRxiv 2019 743989)). Antibodies were validated and titrated under the supervision of a board-certified pathologist (C.M.S.).

CODEX multiplexed tissue staining and imaging. CODEX experiments were performed as previously described. Briefly, coverslips were deparaffinized, rehydrated and heat-induced epitope retrieval was performed using Dako target retrieval solution, pH 9 (Agilent) at 97° C. for 10 min. Each coverslip was stained with an antibody cocktail in a volume of 100 μl overnight at 4° C. in a sealed humidity chamber on a shaker. After multiple fixation steps using 1.6% paraformaldehyde, 100% methanol and BS3 (Thermo Fisher), coverslips were mounted onto acrylic plates and imaged with a Keyence BZ-X710 inverted fluorescence microscope, equipped with a CFI Plan Apo λ 20×/0.75 objective (Nikon), an Akoya CODEX instrument and CODEX driver software (Akoya Biosciences). At the conclusion of the CODEX experiment, H&E stainings were performed and imaged in brightfield mode.

Data processing of CODEX images. Raw TIFF image files were processed using the CODEX Toolkit Uploader, as previously described (Schurch et al., bioRxiv 2019 743989). After processing, the staining quality for each antibody was visually assessed in each tissue microarray spot and segmentation was performed using the DRAQ5 nuclear stain. Marker expression was quantified and Flow Cytometry Standard (FCS) files were imported into CellEngine for cleanup gating. This resulted in a total of 117,220 cells across all tissue microarray spots.

The resulting FCS files were imported into VorteX Clustering Software 39 and subjected to unsupervised X-shirt clustering using an angular distance algorithm. Clustering was based on all antibody markers except: CD11b, CD16, CD164, CCR4, CCR6, EGFR, and p53. The optimal cluster number was guided by the elbow point validation tool in VorteX, resulting in 78 clusters. Clusters were manually verified and assigned to cell types is based on morphology in H&E and fluorescent CODEX images and on their marker expression profiles. Clusters with similar features were merged, resulting in 21 final clusters. The expression frequencies of Ki-67 and select checkpoint molecules (i.e., ICOS, IDO and PD-1) were determined for the resulting T cell clusters by manual gating in CellEngine, with comparison to the raw fluorescent image for each tissue microarray spot.

Cellular neighborhood identification. Cellular neighborhood (CN) identification was performed using a custom k-nearest neighbors' algorithm in Python (Schurch et al, bioRxiv 2019 743989). For each of the 117,220 cells in this experiment, the window size was set at 10, capturing the center cell and its 10 nearest neighboring cells, as measured by the Euclidean distance between X/Y coordinates. To identify 10 CNs, these windows were then clustered by the composition of their microenvironment with respect to the 21 cell types that were previously identified. This resulted in a vector for each window containing the frequency of each of the 21 cell types amongst the 10 neighborhoods. These windows were then clustered using Python's scikit-learn implementation of MiniBatchKMeans with k=10. Each cell was then allocated to the same CN as the window in which it was centered. All CN assignments were validated by overlaying them on the original fluorescent and H&E stained images.

Calculation of spatial distances and ratios between cell types. The X/Y coordinates for each cell type were determined during cellular segmentation, as described above. The minimal distance between each cell type and its nearest other cell types, and the averages of these minimal distances per tissue spot, were calculated in R. To assess whether the average minimal distances between a cell type (CT1) and another cell type (CT2) were significantly different from the average minimal distances of a random sample, the following was performed on a per spot basis. First, the number of CT1 cells was determined. Next, the same number of non-CT1 cells was randomly selected and their minimal distances to CT2 cells was calculated. This was repeated 1000 times, and the average of the random samples was determined. Then the z-score and quantile of the actual average distance relative to the average of the random sample were used as measures of significance.

Given our interest in the relationship of cell distances between three cell types, i.e., effector T cells (CT1), tumor cells (CT2) and Tregs (CT3), the ratio of the minimal distances between CT1-CT2/CT1-CT3 was calculated. To assess whether these ratios were significantly different from those of a random sample, two approaches on a per spot basis were performed. In the first method, for the number of CT1 cells in each spot, the same number of non-CT1 (nCT1) cells was randomly selected. For each of these nCT1 cells, the is ratio of the minimal distances (nCT1-CT2/nCT1-CT3) was calculated and the mean of this sample was determined. This random sampling was repeated 100 times, and the average of all the means was reported. In the second method, the cell type labels (rCT) were randomized in the per spot matrix of distances between each cell. Next, the rCT1 cells and their closest rCT2 cell and rCT3 cells were identified. The rCT1-rCT2/rCT1-rCT3 distance ratios were then calculated and the mean of the sample was determined. This procedure was repeated 100 times and the average of all the means was reported.

Statistical analysis. Frequencies of immune populations were compared with the non-parametric Mann-Whitney-Wilcoxon test using Graphpad Prism. Controlling for multiple comparisons was accomplished with the one-way ANOVA.

### Results

Predictive Biomarker Discovery in CTCL Patients Treated with Pembrolizumab

The PD-1 signaling pathway is dysregulated in CTCL^{7,9,16,17}, and is therefore an attractive therapeutic target. However, only a subset of patients benefit from anti-PD-1 immunotherapy^{8,11}. To identify predictive biomarkers of pembrolizumab response, advanced stage CTCL patients were studied. The patients were from the CITN-10 clinical trial, which enrolled 24 patients with the most common disease subtypes, mycosis fungoides and Sézary syndrome¹¹. All patients in this study had previously failed at least one systemic therapy and were treated with pembrolizumab every 3 weeks for up to 2 years. Traditional immunohistochemistry (IHC) of tumor tissue, as well as gene expression profiling and mass cytometry from the peripheral blood failed to identify any predictive biomarkers in these patients¹¹.

Matched pre- and post-treatment formalin-fixed, paraffin-embedded (FFPE) biopsy material were obtained from 14 CTCL patients, seven pembrolizumab responders and seven non-responders (FIG. 1a.1). Within this cohort, overall survival was significantly longer in responders compared to non-responders (FIG. 1b); no other differences in patient characteristics were observed. As previously reported for this cohort¹¹, traditional IHC showed no differences in tissue expression of T cell, M2 macrophage and PD-1 signaling pathway markers between responders and non-responders (FIG. 1c-d).

To deeply profile the spatial inter-cellular organization and TME architecture in this cohort, an FFPE tissue microarray was created from the most infiltrated areas of the CTCL skin biopsies before and after treatment (FIG. 1a.2). CODEX highly multiplexed tissue imaging and RNAseq was used to interrogate the frequencies, localization, spatial relationships, and functions of the cell types present within the CTCL TME (FIG. 1a.3), and computationally integrated the data in multiple dimensions to discover potential biomarkers of pembrolizumab response (FIG. 1a.4).

So far, traditional tissue imaging methods have failed to definitively discriminate CTCL CD4⁺ tumor cells from non-malignant reactive CD4⁺ T cells at the single-cell level in situ. Additionally, there is no single IHC antibody that is specific for CTCL tumor cells. While CTCL tumor cells express clonally rearranged T cell receptors (TCRs), many benign infiltrating T cells are also clonal, which precludes differentiation by histopathology or clonality analysis¹⁸. Due to its highly multiplexed capability, CODEX is well suited to overcome this challenge and simultaneously characterize the cellular composition and spatial organization of the CTCL TME. Using a panel of 55 markers (FIG. 1e), unsupervised machine learning and manual curation based on marker expression profiles, tissue localization and morphology, 21 unique cell type clusters were clustered (FIG. 1f). These included tumor cell and reactive immune cell subsets as well as stromal, vascular and epithelial cell types. To further verify the assignment of tumor vs. non-malignant reactive CD4⁺ T cell clusters, the relative expression of select markers were compared, which showed loss of CD7, overexpression of CD25 and increased proliferation (Ki-67 positivity) in tumor cells (FIG. 1g-h)¹⁹. The nuclear size of tumor cells was also increased compared to non-malignant reactive CD4+ T cells (FIG. 1i), which is confirmed visually in the DRAQ5 insert of panel g and is consistent with previous reports²⁰. CD4+ T cells and tumor cells were also distinguished with RNAseq (FIG. 1j), with enrichment in known CTCL genes, such as CD27, IL-32, CXCL13, BATF, and TIGIT in tumor cells, irrespective of CD4+ T cells.

### Deep Profiling of the CTCL TME by CODEX

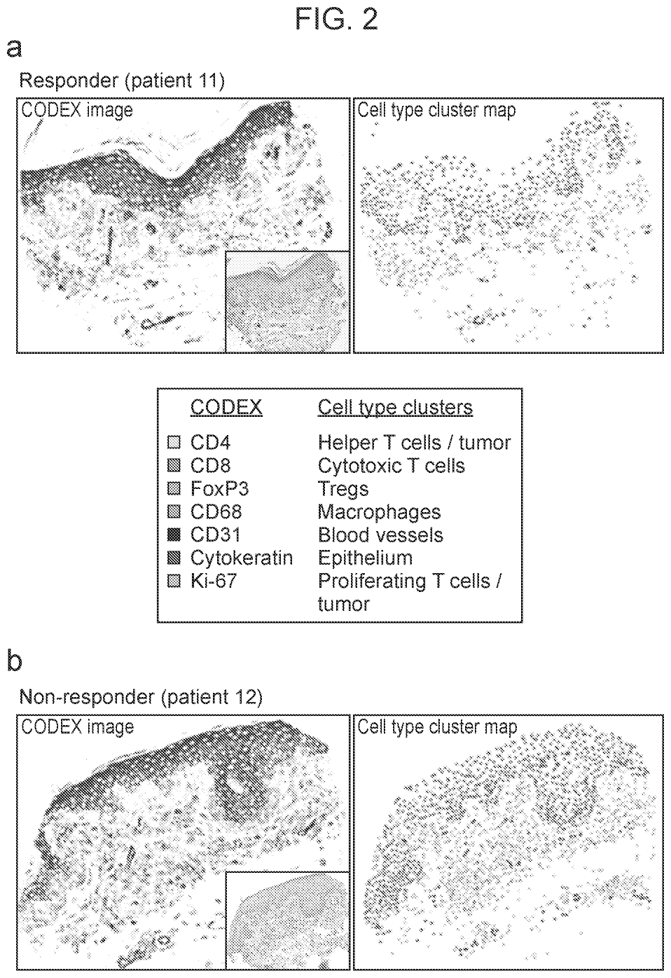

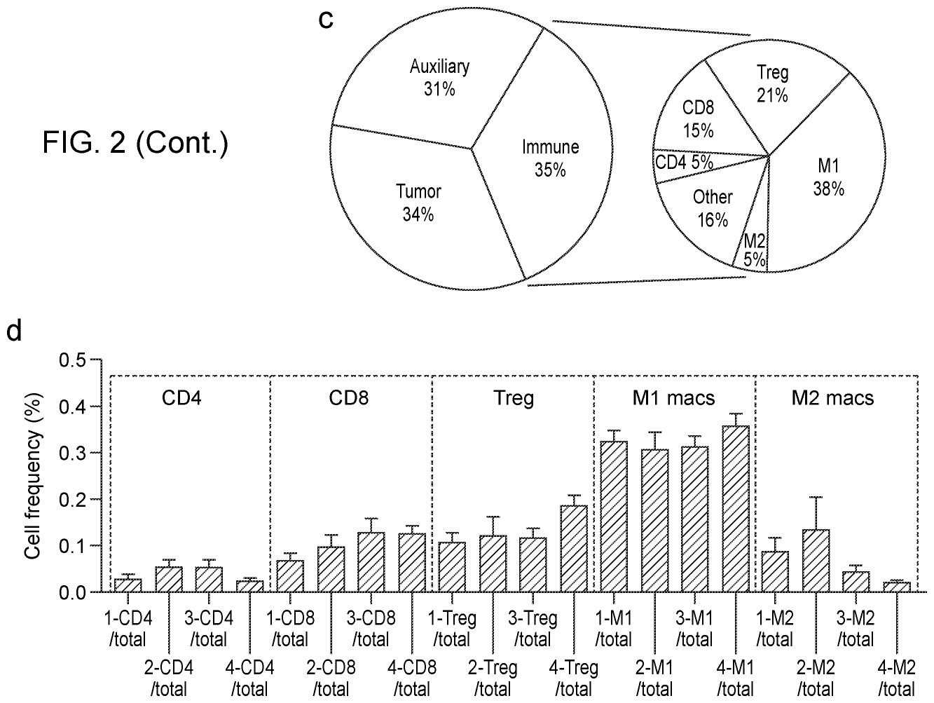

With the ability to discriminate CD4⁺ tumor cells from non-malignant reactive CD4⁺ T cells at the single-cell level, CODEX was used to deeply phenotype the CTCL TME in pembrolizumab responders and non-responders before and after treatment. To visually assess the cellular composition of the TME and simultaneously verify the clustering results within the tissue, seven-color fluorescent overlay images were compared with the corresponding cell type cluster maps for a representative responder and non-responder pre-treatment (FIG. 2a-b). Using select markers for key cell types, including T cell subsets, macrophages, tumor cells, vasculature, and epithelium, excellent visual correlation between CODEX images (FIG. 2a-b, left panels), H&E images (FIG. 2a-b, insets) and cluster maps (FIG. 2a-b, right panels) was observed.

To investigate the interplay between tumor and immune cells, each tissue microarray is core was drilled in the most densely infiltrated area of the corresponding skin biopsy. As such, no significant differences in the frequency of tumor, immune or auxiliary cell types were noted between tissue microarray cores from responders and non-responders in the pre- or post-treatment states. The combined frequencies of tumor, immune and auxiliary cell types were evenly distributed (FIG. 2c, left panel). Within the immune compartment, frequencies were high for M1 macrophages (38%), Tregs (21%) and CD8+ T cells (15%), medium for M2 macrophages (5%) and CD4+ T cells (5%), and low (<5%) for other immune cells, including B cells, plasma cells, dendritic cells, Langerhans cells, mast cells, and neutrophils, which were grouped together (FIG. 2c, right panel). Comparing the frequencies of T cell and macrophage subsets revealed no differences in responders vs. non-responders pre- or post-treatment (FIG. 2d), consistent with the results from traditional IHC pre-treatment (FIG. 1d).

The balance between activated and inhibitory states among tumor-infiltrating T cells is critical for an effective antitumoral immune response²¹. Therefore, to investigate the functional states of in the CTCL TME in more detail, the frequencies of T cells expressing 30 key activation, co-stimulatory, checkpoint, and exhaustion molecules were examined by manually gating the expression levels of ICOS, IDO-1, Ki-67, and PD-1 on T cell subsets, and responders were shown to have a more activated phenotype, whereas non-responders were shown to have a more immunosuppressed phenotype (data not shown). Similar trends were observed in the RNAseq studies (data not shown). Notably, no differences were seen between patient groups in PD-1V tumor, CD4+ or CD8+ T cells.

### Cellular Neighborhoods Reveal Spatial Relationships Predictive of Pembrolizumab Response in CTCL

The architectural features of tumors can have substantial clinical and prognostic utility²²; yet, the mechanisms by which the three-dimensional cellular organization impact antitumoral immunity and immunotherapy outcomes is still poorly characterized. To interrogate the cellular spatial relationships in CTCL before and after pembrolizumab treatment requires that the tissue be examined beyond the level of single cells and simple pairwise cell-cell interactions. To do this, cellular neighborhood (CN) analysis was performed. Briefly, CN analysis integrates data on cellular phenotype and localization to identify unique regions and sub-structures within the tissue. The concept of CNs is analogous to urban neighborhoods, which are defined as geographically localized areas/communities within a larger city that facilitate social interaction²³. Likewise, CNs are is defined by the local composition of similar cell types within the tissue that mediate cell-cell interactions and overarching tissue functions.

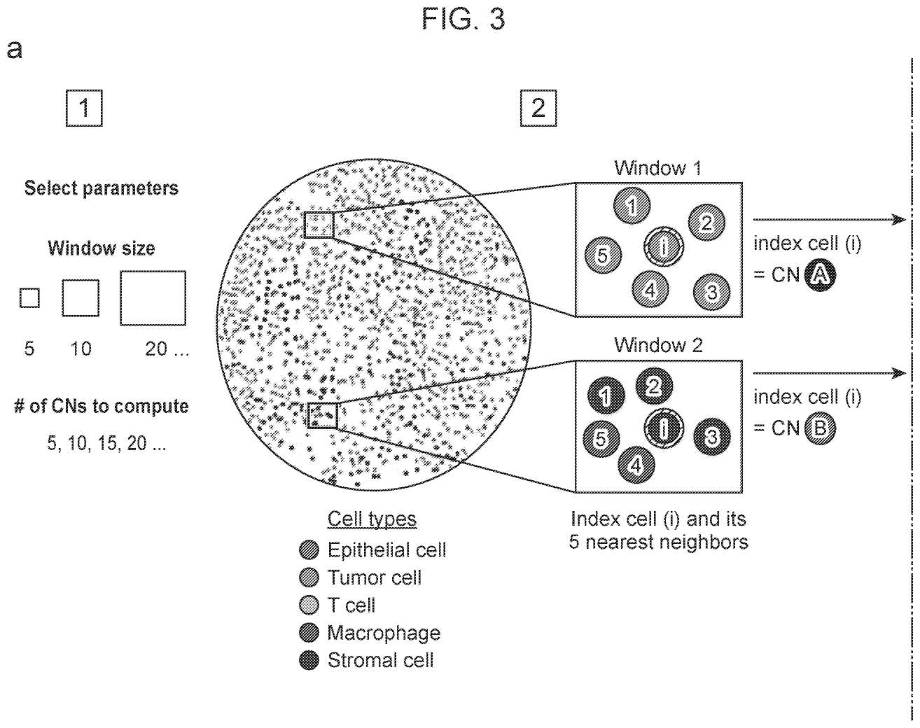

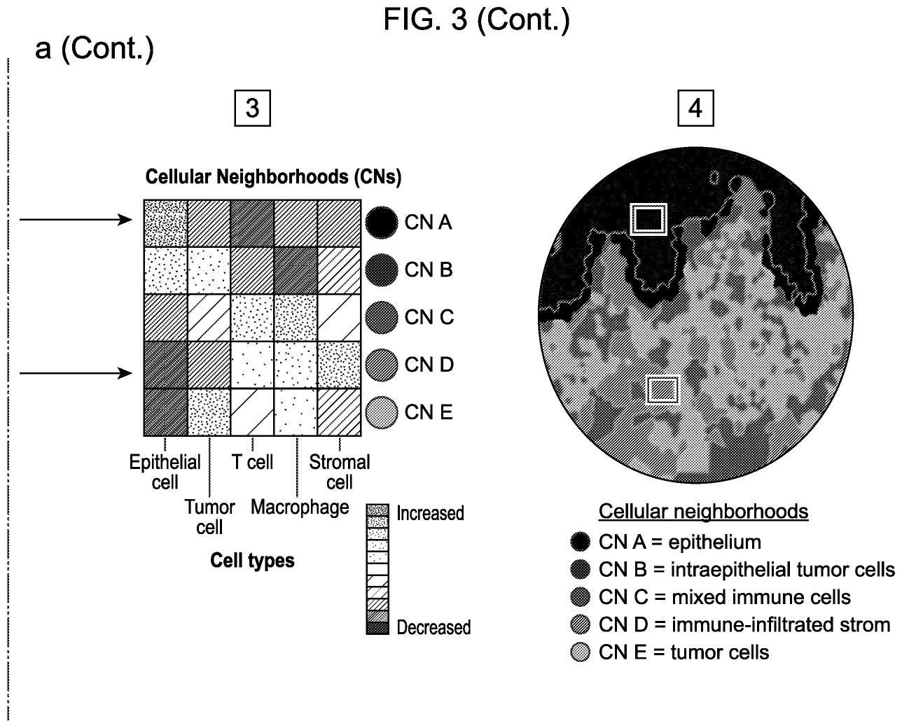

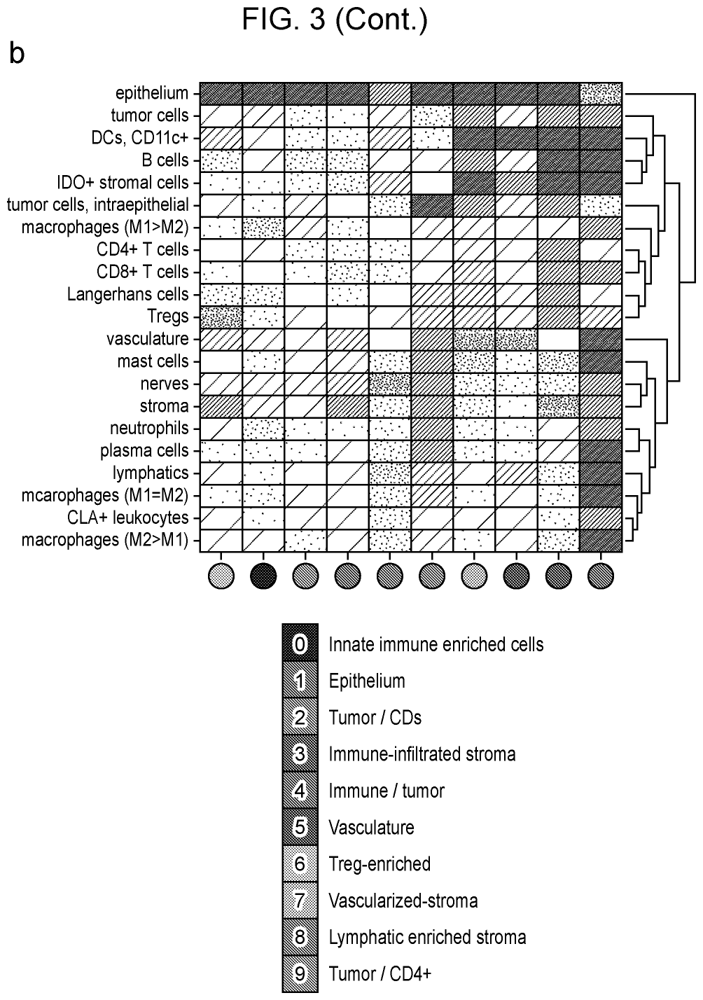

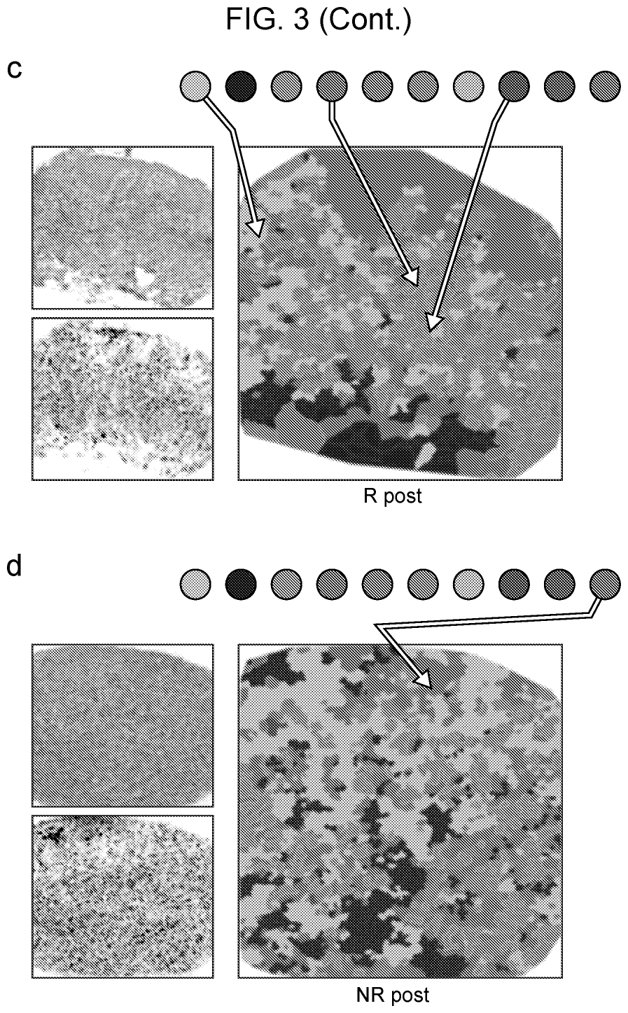

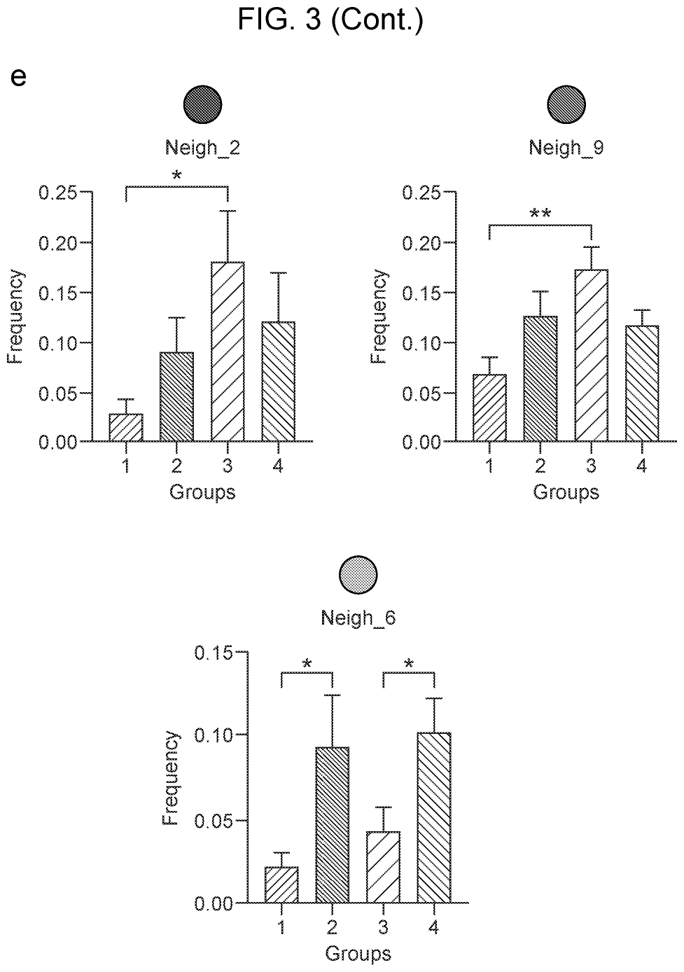

A schematic detailing CN analysis is presented in FIG. 3a. First, key computational parameters are selected, including the window size and the number of CNs to be computed (FIG. 3a.1). In this schematic, a CN window size of five is selected and five CNs are computed. Thus, a central index cell and its five nearest neighbors are assessed (FIG. 3a.2). Based on the cell type composition of its five nearest spatial neighbors, the index cell (i, center) is assigned to a given CN. This analysis is performed for every cell in the tissue, resulting in a heatmap that displays the composition of each CN as a function of cell type frequencies (FIG. 3a.3). Finally, CNs are visualized as Voronoi diagrams, with each CN represented as a distinct color (FIG. 3a.4). Thus, CN analysis extracts quantitative information on the composition and spatial distribution of individual cells to reveal how local cellular niches are organized within the overarching tissue structure. This approach enables deep profiling of the TME architecture across cancer clinical trial cohorts, thereby facilitating the development of prognostic spatial patterns indicative of immunotherapeutic success.

Ten distinct CNs were identified in the cohort, which were conserved across patient groups (FIG. 3b). These CNs recapitulated key tissue components clearly visible in H&E and fluorescent images, such as the epithelium (CN-1) and regions of vasculature (CN-5) (FIG. 3c, green and brown regions, respectively). Furthermore, this method revealed sub-structures within the dermal infiltrate that were not appreciable in H&E or fluorescent images (FIG. 3d), such as regions co-enriched in tumor cells and specific immune cell types, including a tumor/dendritic cell (DC) CN (CN-2), a tumor/CD4+ T cell CN (CN-9), and a tumor/immune CN (CN-4) (FIG. 3c). Additional CNs identified were innate immune cell-enriched (CN-0), Treg-enriched (CN-6), and a stromal CNs rich in immune cells (CN-3), vasculature (CN-7) or lymphatics (CN-8) (FIG. 3c).

While all CNs were represented in responders and non-responders pre- and post-treatment, significant differences in the frequencies of some CNs were observed between patient groups (FIG. 3e). In line with observation of a more activated immune phenotype in responders, the frequencies of the tumor/DC CN (CN-2) and the tumor/CD4+ T cell CN (CN-9) were significantly increased after pembrolizumab treatment. This increase in CN-2 and CN-9 was not observed in non-responders. In contrast, the frequency of the Treg-enriched CN (CN-6) was significantly higher in non-responders compared to responders, both pre- and post-treatment, consistent with an immunosuppressed phenotype. Importantly, is these differences in CN frequencies between patient groups were present despite no differences in the global tissue frequencies of tumor cell or immune cell type frequencies (including CD4+ T cells and Tregs) (FIG. 2c-d). These findings indicate that the tissue's spatial configuration, and not the frequencies of cell types within it, is a key determinant of immunotherapy outcome in this cohort.

### Spatial Ratio Between Tumor Cells, PD-1+CD4+ T Cells and Tregs Predicts Pembrolizumab Response in CTCL

Given the higher abundance of the Treg-enriched CN (CN-6) in non-responders pre- and post-treatment as well as the significant increase of the tumor/CD4+ T cell CN (CN-9) in responders after treatment (FIG. 3), the spatial distribution of these cell types was examined. In the context of pembrolizumab therapy, PD-1+CD4+ T cells, Tregs and tumor cells were examined. Notably, the PD-1+CD4+ T cell frequencies were not different between patient groups or in CN-9. This indicates that spatial organization, as opposed to cell type abundance, is a key driver of immunotherapy outcome. These findings prompted us to investigate the possibility of a simplified predictive model that incorporates the spatial relationships between specific cell types and could be used in a clinical setting.

Using the X/Y positions of each cell, the Euclidean distances between every PD-1+CD4+ T cell and its nearest tumor cell (distance A) and its nearest Treg (distance B) (FIG. 4a, left panel) was measured. Then a SpatialScore was calculated by calculating the ratio of distance A over distance B. When the SpatialScore is a lower value, this implies increased anti-tumor activity (i.e., PD-1+CD4+ T cells are closer to tumor cells and farther from Tregs) (FIG. 4a.1). In contrast, when the SpatialScore is a higher value, this implies increased immunosuppression (i.e., PD-1+CD4+ T cells are closer to Tregs and father from tumor cells) (FIG. 4a.2). The SpatialScore was calculated per cell for each patient group: when less than 0.5, the SpatialScore predicted a successful response to pembrolizumab in CTCL with 80% accuracy (FIG. 4b). The lower SpatialScore seen in responders indicates increased anti-tumor activity, whereas the higher SpatialScore seen in non-responders indicates increased immunosuppression (FIG. 4b, bottom panel), consistent with our earlier findings (FIG. 3d). Notably, these findings become more pronounced in both responders and non-responders after pembrolizumab treatment (FIG. 4b, top panel). It is speculated that these differences in the functional immune phenotypes between responders and non-responders, facilitate the PD-1+CD4+ T cell activation in responders and lead to PD-1+CD4+ T cell suppression in non-responders upon treatment with pembrolizumab. It is important to point out that the SpatialScore does not simply reflect the number of Tregs, PD-1+CD4+ T cells or tumor is cells, as no correlation was identified for any of these cell types relative to the spatial ratio.

### CXCL13 is a Key Driver of Pembrolizumab Response in CTCL

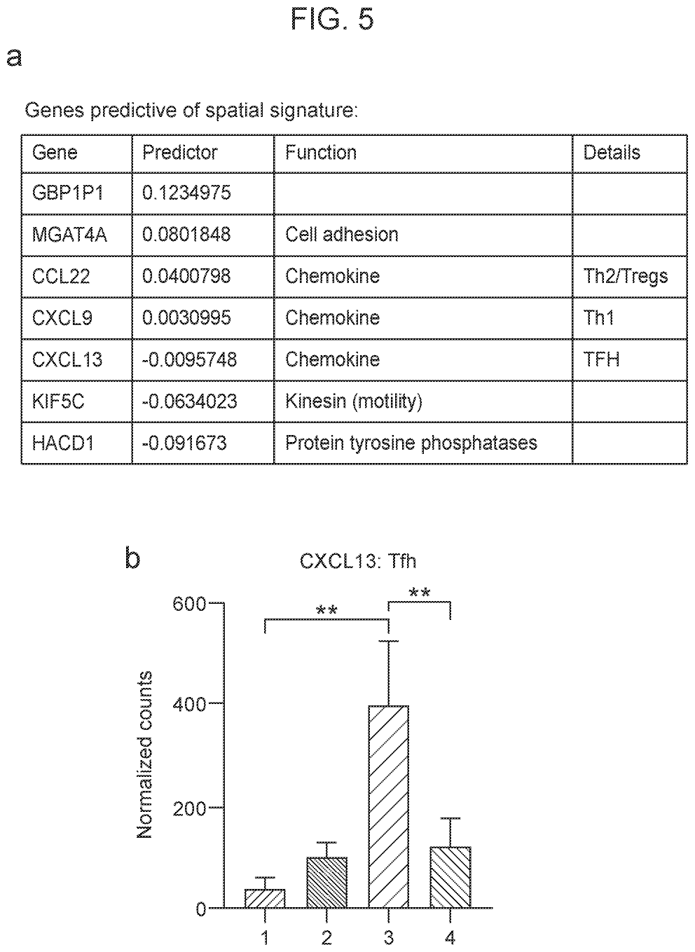

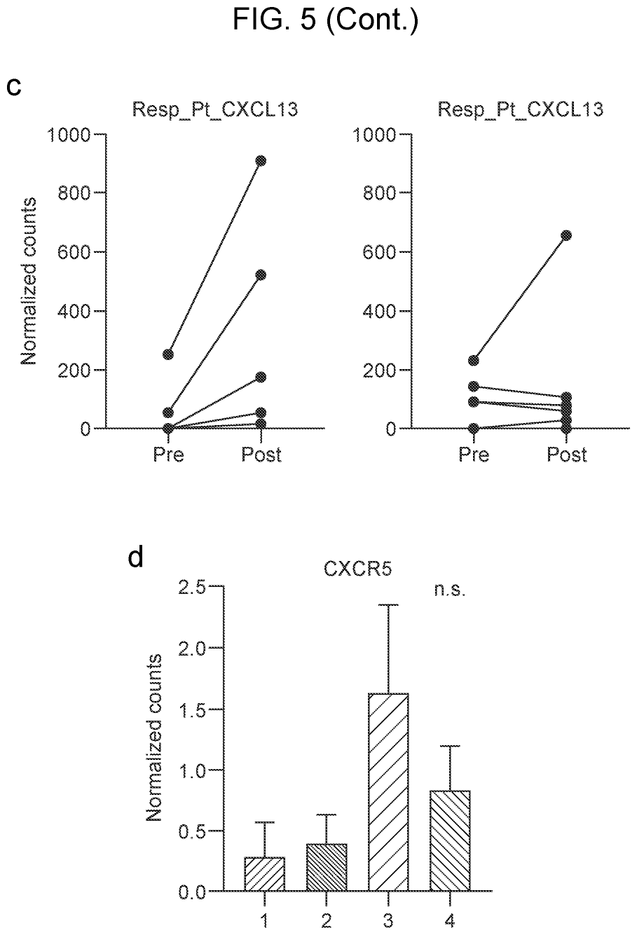

Genes predictive of the SpatialScore were identified Using integrative analysis and seven predictive genes were revealed (FIG. 5a), including CXCL13, which is a chemokine known to be secreted by CTCL tumor cells. Interestingly, CXCL13 expression is significantly increased after pembrolizumab treatment in responders (FIG. 5b). On a per patient basis, CXCL13 expression increased in 5/5 (100%) responders after treatment (FIG. 5c, left), in contrast to 1/6 (16.7%) non-responder patients (FIG. 5c, right). It is also important to point out that CXCR5, which is the receptor for CXCL13, increased in responders post-treatment, but did not reach statistical significance (FIG. 5d).

### Model

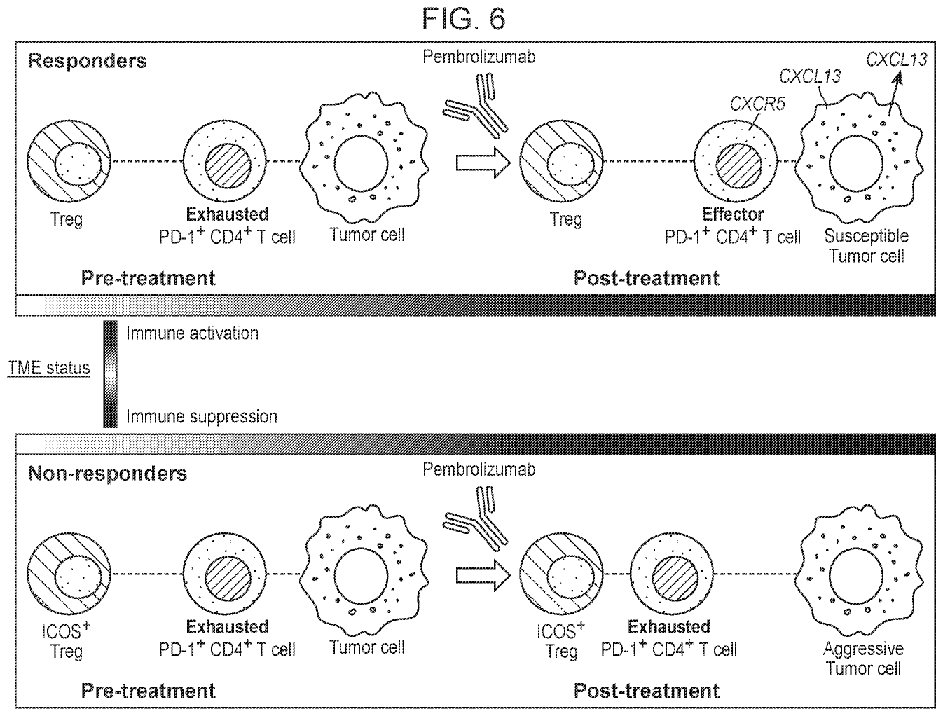

FIG. 6 shows a model of the key cell types and the how their positioning is associated with immunotherapy outcome

### REFERENCES

1 Kim, Y. H., Liu, H. L., Mraz-Gernhard, S., Varghese, A. & Hoppe, R. T. Long-term outcome of 525 patients with mycosis fungoides and Sezary syndrome: clinical prognostic factors and risk for disease progression. Arch Dermatol 139, 857-866, doi:10.1001/archderm.139.7.857 (2003).
2 Willemze, R. et al. WHO-EORTC classification for cutaneous lymphomas. Blood 105, 3768-3785, doi:10.1182/blood-2004-09-3502 (2005).
3 Wilcox, R. A. Mogamulizumab: 2 birds, 1 stone. Blood 125, 1847-1848, doi:10.1182/blood-2015-02-625251 (2015).
4 Gong, J., Chehrazi-Raffle, A., Reddi, S. & Salgia, R. Development of PD-1 and PD-L1 inhibitors as a form of cancer immunotherapy: a comprehensive review of registration trials and future considerations. J Immunother Cancer 6, 8, doi:10.1186/s40425-018-0316-z (2018).
5 Sharpe, A. H. & Pauken, K. E. The diverse functions of the PD1 inhibitory pathway. Nat Rev Immunol 18, 153-167, doi:10.1038/nri.2017.108 (2018).
6 Ribas, A. Tumor immunotherapy directed at PD-1. N Engl J Med 366, 2517-2519, doi:10.1056/NEJMe1205943 (2012).
7 Ungewickell, A. et al. Genomic analysis of mycosis fungoides and Sezary syndrome identifies recurrent alterations in TNFR2. Nat Genet 47, 1056-1060, doi:10.1038/ng.3370 (2015).
8 Lesokhin, A. M. et al. Nivolumab in Patients With Relapsed or Refractory Hematologic Malignancy: Preliminary Results of a Phase Ib Study. J Clin Oncol 34, 2698-2704, doi:10.1200/JCO.2015.65.9789 (2016).
9 Kantekure, K. et al. Expression patterns of the immunosuppressive proteins PD-1/CD279 and PD-L1/CD274 at different stages of cutaneous T-cell lymphoma/mycosis fungoides. Am J Dermatopathol 34, 126-128, doi:10.1097/DAD.0b013e31821c35cb (2012).
10 Querfeld, C. et al. Primary T Cells from Cutaneous T-cell Lymphoma Skin Explants Display an Exhausted Immune Checkpoint Profile. Cancer Immunol Res 6, 900-909, doi:10.1158/2326-6066.CIR-17-0270 (2018).
11 Khodadoust, M. S. et al. Pembrolizumab in Relapsed and Refractory Mycosis Fungoides and Sezary Syndrome: A Multicenter Phase II Study. J Clin Oncol 38, 20-28, doi:10.1200/JCO.19.01056 (2020).
12 Wartewig, T. et al. PD-1 is a haploinsufficient suppressor of T cell lymphomagenesis. Nature 552, 121-125, doi:10.1038/nature24649 (2017).
13 Wartewig, T. & Ruland, J. PD-1 Tumor Suppressor Signaling in T Cell Lymphomas. Trends Immunol 40, 403-414, doi:10.1016/j.it.2019.03.005 (2019).
14 Postow, M. A., Callahan, M. K. & Wolchok, J. D. Immune Checkpoint Blockade in Cancer Therapy. J Clin Oncol 33, 1974-1982, doi:10.1200/JCO.2014.59.4358 (2015).
15 Ribas, A. & Wolchok, J. D. Cancer immunotherapy using checkpoint blockade. Science 359, 1350-1355, doi:10.1126/science.aar4060 (2018).
16 Choi, J. et al. Genomic landscape of cutaneous T cell lymphoma. Nat Genet 47, 1011-1019, doi:10.1038/ng.3356 (2015).
17 Wang, L. et al. Genomic profiling of Sezary syndrome identifies alterations of key T cell signaling and differentiation genes. Nat Genet 47, 1426-1434, doi:10.1038/ng.3444 (2015).
18 de Masson, A. et al. High-throughput sequencing of the T cell receptor beta gene identifies aggressive early-stage mycosis fungoides. Sci Transl Med 10, doi:10.1126/scitranslmed.aar5894 (2018).
19 Swerdllow, S. H. et al. in WHO Classification of Tumours, Revised 4th Edition, Volume 2 Vol. 2 (IARC Press, Lyon, 2017).
20 Iwahara, K. & Hashimoto, K. T-cell subsets and nuclear contour index of skin-infiltrating T-cells in cutaneous T-cell lymphoma. Cancer 54, 440-446, doi:10.1002/1097-0142(19840801)54:3<440::aid-cncr2820540311>3.0.co;2-m (1984).
21 Wherry, E. J. T cell exhaustion. Nat Immunol 12, 492-499 (2011).
22 Sautes-Fridman, C. et al. Tertiary Lymphoid Structures in Cancers: Prognostic Value, Regulation, and Manipulation for Therapeutic Intervention. Front Immunol 7, 407, doi:10.3389/fimmu.2016.00407 (2016).
23 Schuck, A. M. a. R., Dennis P. in Community Change: Theories, Practice, and Evidence Promoting change through community level interventions Ch. 2, 61-140 (The Aspen Institute, 2006).
24 Zhang, X., Kelaria, S., Kerstetter, J. & Wang, J. The functional and prognostic implications of regulatory T cells in colorectal carcinoma. J Gastrointest Oncol 6, 307-313, doi:10.3978/j.issn.2078-6891.2015.017 (2015).
25 Haabeth, O. A. et al. How Do CD4(+) T Cells Detect and Eliminate Tumor Cells That Either Lack or Express MHC Class II Molecules? Front Immunol 5, 174, doi:10.3389/fimmu.2014.00174 (2014).
26 Kitano, S. et al. Enhancement of tumor-reactive cytotoxic CD4+ T cell responses after ipilimumab treatment in four advanced melanoma patients. Cancer Immunol Res 1, 235-244, doi:10.1158/2326-6066.CIR-13-0068 (2013).
27 Togashi, Y., Shitara, K. & Nishikawa, H. Regulatory T cells in cancer immunosuppression-implications for anticancer therapy. Nat Rev Clin Oncol 16, 356-371, doi:10.1038/s41571-019-0175-7 (2019).
28 Tang, H., Qiao, J. & Fu, Y. X. Immunotherapy and tumor microenvironment. Cancer Lett 370, 85-90, doi:10.1016/j.canlet.2015.10.009 (2016).
29 Nagarsheth, N., Wicha, M. S. & Zou, W. Chemokines in the cancer microenvironment and their relevance in cancer immunotherapy. Nat Rev Immunol 17, 559-572, doi:10.1038/nri.2017.49 (2017).
30 Binnewies, M. et al. Understanding the tumor immune microenvironment (TIME) for effective therapy. Nat Med 24, 541-550, doi:10.1038/s41591-018-0014-x (2018).
31 Melero, I. et al. Evolving synergistic combinations of targeted immunotherapies to combat cancer. Nat Rev Cancer 15, 457-472, doi:10.1038/nrc3973 (2015).
32 Smyth, M. J., Ngiow, S. F., Ribas, A. & Teng, M. W. Combination cancer immunotherapies tailored to the tumour microenvironment. Nat Rev Clin Oncol 13, 143-158, doi:10.1038/nrclinonc.2015.209 (2016).
33 Church, S. E. & Galon, J. Tumor Microenvironment and Immunotherapy: The Whole Picture Is Better Than a Glimpse. Immunity 43, 631-633, doi:10.1016/j.immuni.2015.10.004 (2015).
34 Taube, J. M. et al. Association of PD-1, PD-1 ligands, and other features of the tumor immune microenvironment with response to anti-PD-1 therapy. Clin Cancer Res 20, 5064-5074, doi:10.1158/1078-0432.CCR-13-3271 (2014).
35 Michot, J. M. et al. Immune-related adverse events with immune checkpoint blockade: a comprehensive review. Eur J Cancer 54, 139-148, doi:10.1016/j.ejca.2015.11.016 (2016).
36 Ghate, S. R., Li, Z., Tang, J. & Nakasato, A. R. Economic Burden of Adverse Events Associated with Immunotherapy and Targeted Therapy for Metastatic Melanoma in the Elderly. Am Health Drug Benefits 11, 334-343 (2018).
37 Olsen, E. A. et al. Clinical end points and response criteria in mycosis fungoides and Sezary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol 29, 2598-2607, doi:10.1200/JCO.2010.32.0630 (2011).
38 Basu, A., Yearley, J. H., Annamalai, L., Pryzbycin, C. & Rini, B. Association of PD-L1, PD-L2, and Immune Response Markers in Matched Renal Clear Cell Carcinoma Primary and Metastatic Tissue Specimens. Am J Clin Pathol 151, 217-225, doi:10.1093/ajcp/agy141 (2019).
39 Samusik, N., Good, Z., Spitzer, M. H., Davis, K. L. & Nolan, G. P. Automated mapping of phenotype space with single-cell data. Nat Methods 13, 493-496, doi:10.1038/nmeth.3863 (2016).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the s appended claims.

1. A method for predicting response to immunotherapy, comprising:
(a) performing a multiplexed binding assay on a tissue section of a tumor obtained from a cancer patient to identify at least: (i) cancer cells, (ii) effector immune cells and (iii) immunosuppressive cells in the tissue section;
(b) measuring, for each cell of a plurality of the effector immune cells:
(i) the physical distance to its most proximal cancer cell; and
(ii) the physical distance to its most proximal immunosuppressive cell; and
(c) calculating, for each of the effector immune cells analyzed in (b), the ratio of the distance measured in step (b)(i) and distance measured in step (b)(ii),
wherein the ratios calculated in (c) are predictive of the patient's response to immunotherapy.

2. The method of claim 1, wherein the cancer cells identified in step (a) are melanoma cells, carcinoma cells, lymphoma cells, sarcoma cells or glioma cells.

3. The method of claim 1, wherein the cancer cells identified in step (a) are:
i. melanoma cells identified by expression of one or more of the following markers: S-100, Melan-A, Sox10, MITF, tyrosinase, and HMB45;
ii. carcinoma cells identified by the expression of one or more of the following markers: pan-cytokeratin (CK), CK7, CK20, CK5/6, CK8/18, napsin A, TTF-1, PSA, PSMA, CDX2, GATA3, synaptophysin, chromogranin A, NSE, EpCAM, and MUC-1;
iii. lymphoma/leukemia cells identified by the expression of one or more of the following markers: CD45, CD3, PAX5, CD20, Myc, CyclinD1, BCL-2, BCL-6, IRF4, CD138, CD30, kappa, lambda, TdT, CD10, ALK, and lysoszyme;
iv. sarcoma/mesothelioma cells identified by the expression of one or more of the following markers: vimentin, SMA, desmin, caldesmin, MyoD1, CD34, calretinin, podoplanin, and CD47;
v. glioma cells/neural tumor cells identified by the expression of one or more of the following markers: GFAP, IDH-1(R132H), neurofilament, and NeuN; or
vi. germ cell tumor cells identified by the expression of one or more of the following markers: beta-HCG, OCT4, SALL4, PLAP, inhibin A, HPL and AFP.

4. The method of claim 1, wherein the effector immune cells identified in step (a) include CD4+ T cells, CD8+ T cells, gamma-delta T cells, NK cells, NK T cells and M1 macrophages.

5. The method of claim 1, wherein the effector immune cells identified in step (a) include:
i. CD4+ T cells identified by the expression of CD3, CD4, and TCR-a/b;
ii. CD8+ T cells identified by the expression of CD3, CD8, TCR-a/b;
iii. gamma-delta T cells identified by the expression of CD3, and TCR-g/d;
iv. NK cells identified by the expression of CD16 and CD56;
v. NK T cells identified by the expression of CD3, CD16, and CD56; and
vi. M1 macrophages identified by the expression of CD68.

6. The method of claim 1, wherein the immunosuppressive cells identified in step (a) include regulatory T cells, M2 macrophages, and N2 granulocytes.

7. The method of claim 1, wherein the immunosuppressive cells identified in step (a) include:
i. regulatory T cells identified by the expression of FoxP3;
ii. M2 macrophages identified by the expression of CD163 and CD206; and
iii. N2 granulocytes identified by the expression of CD15 and MMP9.

8. The method of claim 1, further comprising:
(d) averaging the ratios obtained in (c) to obtain a score.

9. The method of claim 8, further comprising:
(e) administering an immune checkpoint inhibitor to the patient if the score is at or below a threshold.

10. The method of claim 9, wherein the immune checkpoint inhibitor is antibody that binds to CTLA-4, PD1, PD-L1, TIM-3, VISTA, LAG-3, IDO or KIR.

11. The method of claim 1, wherein the plurality of binding agents used in step (a) comprises binding agents that specifically bind to CD3, CD4, CD8, TCR-g/d, CD16, CD56, CD68 (for effector immune cells) and FoxP3, CD163, CD206, CD15, MMP9 (and for immunosuppressive cells).

12. A method of treatment comprising:
(a) receiving a report that provides a score indicating the ratio of the physical distances of effector immune cells to their most proximal cancer cell in a tumor from a patient relative to the physical distances of the effector immune cells to their most proximal immunosuppressive cell in the tumor; and
(b) identifying the patient as a candidate for immunotherapy if the ratio is at or below a threshold.

13. The method of claim 12, further comprising:
(c) administering an immune checkpoint inhibitor to the patient.

14. The method of claim 13, wherein the immune checkpoint inhibitor is antibody that binds to CTLA-4, PD1, PD-L1, TIM-3, VISTA, LAG-3, IDO or KIR.

15. A method for selecting a patient for treatment by an immune checkpoint inhibitor, comprising:
selecting a cancer patient for treatment by an immune checkpoint inhibitor based on the ratio of the physical distances of effector immune cells to their most proximal cancer cell in a tumor from the patient relative to the physical distances of the effector immune cells to their most proximal immunosuppressive cell in the tumor.

16. The method of claim 15, wherein the patient is selected if the ratio is at or below a threshold.
